(19) United States
(12) Patent Application Publication
(10) Pub. No.: US 2023/0107428 A1
(43) Pub. Date: Apr. 6, 2023

(54) 23-O-ACETYLALISOL B AS A NOVEL THERAPEUTIC AGENT FOR CORONAVIRUS INDUCED SEVERE ACUTE RESPIRATORY SYNDROME

(71) Applicant: The University of Hong Kong, Hong Kong (CN)

(72) Inventors: Jiangang Shen, Hong Kong (CN); Qiaohui Du, Hong Kong (CN); Shuofeng Yuan, Hong Kong (CN)

(21) Appl. No.: 17/936,179

(22) Filed: Sep. 28, 2022

Related U.S. Application Data
(60) Provisional application No. 63/261,759, filed on Sep. 28, 2021.

(51) Int. Cl. A61K 31/58; A61P 31/14

(52) U.S. Cl. CPC A61K 31/58; A61P 31/14

(57) ABSTRACT

The subject invention pertains to a potent therapeutic agent, 23-O-Acetylalisol B, as an antiviral drug to treat COVID-19. 23-O-Acetylalisol B can broadly and dose-dependently inhibit coronavirus (CoVs), including MERS-CoV, SARS-CoV-2, SARS-CoV-2 Alpha and Delta variants. 23-O-Acetylalisol B has anti-inflammation and immunomodulation effects and has therapeutic effects to significantly ameliorate the CoV infection-induced lung damage.

### CROSS-REFERENCE TO RELATED APPLICATION

This application claims the benefit of U.S. Ser. No. 63/261,759, filed Sep. 28, 2021, which is hereby incorporated by reference in its entirety including any tables, figures, or drawings.

### BACKGROUND OF THE INVENTION

Within two decades, the world's human population has undergone three major coronavirus (CoV) outbreaks. The COVID-19 pandemic is the third zoonotic CoV outbreak of the century after severe acute respiratory syndrome (SARS) in 2003 and Middle East respiratory syndrome (MERS) in 2012. COVID-19 has become a major public health disaster worldwide. Until Sep. 16, 2022, total 611 million people have been reported to be SARS-CoV-2 positive and 6.52 million people died from COVID-19 in the world. Although COVID-19 vaccinations have been widely used, the COVID-19 pandemic is still out of control globally. Currently, treatment options for CoVs are largely lacking. Thus, development of novel therapeutic drugs is timely important.

COVID-19 in humans has a broad clinical spectrum ranging from mild to severe manifestations, with a mortality rate of ˜2% worldwide [1]. The high transmissibility of SARS-CoV-2 was attributed to a significant proportion of mild or asymptomatic infections [2, 3]. Moreover, due to the high and fast spread of SARS-CoV-2, several mutated CoVs variants have already emerged, and these mutations may alter various aspects of virus biology, such as pathogenicity, infectivity, transmissibility and/or antigenicity [4]. The infection of SARS-CoV-2 and SARS-CoV-2 Delta variant led to 2% of mortality rate, approximately, and the clinical of COVID-19 has a broad spectrum ranging from mild to severe manifestations [5, 6]. With the newly emerged SARS-CoV-2 Omicron variant, the confirmed cases have dramatically increased worldwide. Although COVID-19 vaccination has been widely used, the critical illness ratio in aged patients is still high in Omicron variant. Recent progress indicate that the combination treatment of interleukin-6 receptor inhibitors tocilizumab and sarilumab increased survival in severe patients in the intensive care units [20]. The only widely used antiviral drug Paxlovid (nirmatrelvir plus ritonavir), a SAR-CoV-2 main protease inhibitor, has been proved for antiviral activities in SARS-CoV-2 induced mouse model and phase I clinical trials [21]. A phase 2/3 clinical trials reveal that Paxlovid decreased the risk of hospitalization or death by 89% [22,23]. However, with the most update study, Paxlovid treatment shows greater incidence of viral rebound than untreated Omicron variant infected patients [24]. With enhanced spreading capacity of SARS-CoV-2 Omicron variant, antibody evasion [25] and/or drug resistances [24], seeking effective and safe antiviral treatment for COVID-19 becomes the highest priority.

Therefore, with the daily increase of new cases of COVID-19 infections, the development of novel therapeutic options is urgently needed.

### BRIEF SUMMARY OF THE INVENTION

This invention pertains to a potent therapeutic agent, 23-O-Acetylalisol B, as an antiviral drug to treat COVID-19 and a novel immunomodulation agent for immune disorders. 23-O-Acetylalisol B can have bioactivities that include antivirus, anti-inflammation and immunomodulation to pan-coronavirus infections including MERS-CoV, SARS-CoV-2, SARS-CoV-2 Alpha and Delta variants. 23-O-Acetylalisol B is a nature triterpenoid isolated from medicinal plant Rhizoma alismatis in Traditional Chinese Medicine (TCM). Previous studies revealed that 23-O-Acetylalisol B has anti-inflammation, hepatoprotective, and cardiovascular protective activities through different underlying mechanisms [9-11]. Additionally, it was reported that Alisol O, triterpenoid with similar structure with 23-O-Acetylalisol B, could inhibit hepatitis B virus in vitro [12].

In certain embodiments, 23-O-Acetylalisol B can reduce CoV replication and mutated CoV replication. 23-O-Acetylalisol B can be a potential drug candidate for severe acute respiratory syndrome (SARS) thorough broadly inhibiting CoVs and immunomodulation. In certain other embodiments, 23-O-Acetylalisol B can be an immunomodulation agent for autoimmune disorders, such as, for example, multiple sclerosis, systemic lupus erythematosus and rheumatoid arthritis.

### BRIEF DESCRIPTION OF THE DRAWINGS

The patent or application file contains at least one drawing executed in color. Copies of this patent or patent application publication, with color drawing(s), will be provided by the Office upon request and payment of the necessary fee.

FIGS. 1A-1L. Alisol B inhibits a broad-spectrum of human-pathogenic CoVs in vitro. Caco-2 cell line was infected with CoVs (multiplicity of infection (MOI)=0.1) and treated with alisol B in 4 dosages (10, 20, 30, 40 μM) and remdesivir. (FIG. 1A) Chemical structure of alisol B. (FIGS. 1b-1G) Virus titer in the cell culture supernatant were determined by plaque assay at 24 h.p.i. for MERS-CoV (FIG. 1B) and 48 h.p.i for SARS-CoVs-2 (FIG. 1C), Alpha variant (FIG. 1D), Delta variant (FIG. 1E), Omicron BA.1.1 variant (FIG. 1F), Omicron BA.5.2 variant (FIG. 1G). (FIG. 1H-1L) Intracellular viral loads were tested by qualitative polymerase chain reaction with reverse transcription at 48 h.p.i. and normalized by human β-actin at 24 h.p.i. for MERS-CoV (FIG. 1H) and 48 h.p.i for SARS-CoVs-2 (FIG. 1I), Alpha variant (FIG. 1J), Delta variant (FIG. 1K), Omicron BA.5.2 variant (FIG. 1L). Viral titer and viral copies were normalized with cell viabilities. All the data are presented as mean±S.E.M (*p<0.05, **p<0.01, ***p<0.001, relative to vehicle group, one-way ANOVA followed by Dunnett test). All experiments were performed in n=3-4 biological replicates.

FIGS. 2A-2F. Alisol B inhibits viral entry. FIGS. 2A-2C show the VSV-based pseudotyped viral particle results in which SARS-CoV-2 pseudotyped particles were pre-treated with alisol B, and the mixture was added to A549-ACE2-TMPRSS2 cells (FIG. 2A), VeroE6 cells (FIG. 2B) and VeroE6-TMPRSS2 cells (FIG. 2C) under culture condition for 24 hours. In FIG. 2D, SARS-CoV-2 Omicron variant type pseudotyped viral particle was incubated with alisol B and infected to the HEK249-hACE2 cells. In FIG. 2E, ACE2 activity was detected through binding to RBD of wild type S protein. In FIG. 2F, molecular docking analysis reveals the binding affinity of alisol B to ACE2 and the results are shown as 3D molecular analysis. All the data are presented as mean S.E.M (*p<0.05, **p<0.01, ***p<0.001, relative to vehicle group, dunnett test, one-way ANOVA). All experiments were performed in n=3-4 biological replicates.

FIGS. 3A-3N. Alisol B exhibits antiCoVs activities in vivo. Hamsters (n=4-6) were intranasally inoculated with 10⁴ PFU of SARS-CoV-2 and SARS-CoV-2 Delta variant. Alisol B was intraperitoneal administrated to hamster with either vehicle (ethanol, PEG400 and saline solvent system) or alisol B (60 mg/kg) for three consecutive days. (FIG. 3A and FIG. 3E) Virus copies in the hamster lung tissue. Hamster feces freshly collected at 3 dpi were subjected to SARS-CoV-2 (FIG. 3A) and SARS-CoV-2Delta variant (FIG. 3E) viral copies detection by RT-qPCR assays. (FIG. 3B and FIG. 3F) Viral yield in the hamster lung tissue was titrated by plaque assays after 3 days alisol B treatment in SARS-CoV-2 and the SARS-CoV-2 Delta variant, respectively. (FIG. 3C and FIG. 3G) Representative immunofluorescence images of the viral N protein distribution in lung tissue sections after SARS-CoV-2 (FIG. 3C) infection and SARS-CoV-2 Delta variant (FIG. 3G) infection. (FIG. 3D and FIG. 3H) Quantification of the cells positive with VNP colocalized with nucleus in SARS-CoV-2 and the SARS-CoV-2 Delta variant, respectively. (FIG. 3I and FIG. 3J) Representative images of H&E-stained lung tissue section from SARS-CoV-2 (FIG. 3I) and Delta variant (FIG. 3J) infected hamsters. (FIG. 3K and FIG. 3L) IFN-γ level in SARS-CoV-2 (FIG. 3K) and Delta variant (FIG. 3L) infected hamster serum quantified by ELISA assay. (FIG. 3M) Virus copies in nasal turbinate and lung tissues of Omicron BA.1.1 infected hACE2-mice. (FIG. 3N) Inflammation cytokines detected by ELISA in serum of Omicron infected mice. All the data are presented as mean±S.E.M (*p<0.05, **p<0.01, ***p<0.001, relative to vehicle group, unpaired two-tailed Student's t-test between vehicle and alisol B treatment group. one-way ANOVA followed by Dunnett test for NP⁺ cells). All experiments were performed in n=4-6 biological replicates.

FIGS. 4A-4G. Transcriptional analysis of SARS-CoV-2 and SARS-CoV-2 Delta variant infected hamster lung tissues with alisol B treatment. (FIG. 4A and FIG. 4D) Heat map of DEGs in uninfected and SARS-CoV-2 (FIG. 4A) and Delta variant (FIG. 4D) infected hamster lungs administrated with alisol B or vehicle controls. (FIG. 4B and FIG. 4E) Pathway functional enrichment analysis showing the down-regulated DEGs in the alisol B treatment group compared with vehicle control groups in both SARS-CoV-2 (FIG. 4B) and Delta variant (FIG. 4E) infected hamsters. (FIG. 4C and FIG. 4F) q-PCR assay showing the expression levels of chemokine and cytokine in the lung tissues homogenate from the hamsters infected with SARS-CoV-2 (C) and Delta variant (F) (n=3-4) at 3 d.p.i. All the data are presented as mean±S.E.M (*p<0.05, relative to vehicle group, unpaired two-tailed Student's t-test between vehicle and alisol B treatment group for mRNA expression levels. All experiments were performed in n=3-4 biological replicates). (FIG. 4G) Gene Ontology Biological Process (GO-BP) analysis to compare the differential expressed genes (FDR≤0.05) in Alisol B treatment group and vehicle control group.

FIGS. 5A-5H. Alisol B modulates immune response in SARS-CoV-2 diseases. Hamsters (n=4-6) were intranasally inoculated with 10⁴ PFU of SARS-CoV-2 and SARS-CoV-2 Delta variant. Alisol B was intraperitoneal administration to hamster either vehicle (ethanol, PEG400 and saline solvent system) and alisol B (60 mg/kg) for three consecutive days. (FIG. 5A and FIG. 5C) Representative confocal images of the CD11b positive macrophages in lung tissue sections after SARS-CoV-2 (FIG. 5A) infection and SARS-CoV-2 Delta variant (FIG. 5C) infection. (FIG. 5B and FIG. 5D) Quantification of the density of CD11b positive cells in lung tissues of SARS-CoV-2 (B) and SARS-CoV-2 Delta variant (FIG. 5D) infection. (FIG. 5E and FIG. 5G) Representative confocal images of the CD3 positive T lymphocytes in lung tissue sections after SARS-CoV-2 (FIG. 5E) infection and SARS-CoV-2 Delta variant (FIG. 5G) infection. (FIG. 5F and FIG. 5H) Quantification of the density of CD3 positive cells in lung tissue sections after SARS-CoV-2 (FIG. 5F) infection and SARS-CoV-2 Delta variant (FIG. 5H) infection. All the data are presented as mean±S.E.M (*p<0.05, **p<0.01, ***p<0.001, relative to vehicle group, Dunnett test, one-way ANOVA). All the quantification were conducted based on 2 views in one sample.

FIGS. 6A-6N. Alisol B suppressed pro-inflammatory immune cells activation and cytokines release in human lymphocytes. (FIG. 6A) The proliferation of CD4⁺ T cells was inhibited after 48 h alisol B treatment dose-dependently. (FIG. 6B) IL-17 was assessment in cell culture supernatant of Th17 cells inducted by alisol B and cytokines. (FIG. 6C and FIG. 6H) Representative flow cytometry results of CD169 (FIG. 6C) and CD11b (FIG. 6H) positive cells treated with alisol B and DMSO as control. (FIGS. 6D-6G and FIGS. 6J-6M) Quantification of the percentage of CD169 (FIGS. 6D-6G) and CD11b (FIG. 6H) positive cells in alisol B treated macrophages. (FIG. 6N and FIG. 6I) Representative results of flow cytometry and quantification of the B220⁺ cells after 48 h alisol B treatment. All the data are presented as mean±S.E.M (*p<0.05, **p<0.01, ***p<0.001, relative to vehicle group, Dunnett test, one-way ANOVA). All experiments were performed in triplicate and repeated twice for confirmation.

FIG. 7A-7M. Alisol B suppressed pro-inflammatory immune cells activation and cytokines release in mice lymphocytes. (FIGS. 7A-7C and FIGS. 7F-71I) Representative flow cytometry results of IL17+ T cells (FIGS. 7A-7C) and IFNγ+ T cells (FIGS. 7F-71I) treated with alisol B and DMSO as control. (FIG. 7D and FIG. 71) Quantification of the percentage of IL17⁺ T cells (FIG. 7D) and IFNγ⁺ T cells (FIG. 71) positive cells in alisol B treated T cells. (FIG. 7E and FIG. 7J) IL-17 (FIG. 7E) and IFNγ (FIG. 7J) was assessment in cell culture supernatant after 48 h alisol B treatment. (FIG. 7K and FIG. 71) Relative intensity of iNOS in CD68 and CD40 dual positive M1 macrophages. (FIG. 7M) TNFα content was detected in culture supernatant. All the data are presented as mean±S.E.M (*p<0.05, **p<0.01, ***p<0.001, relative to vehicle group, Dunnett test, one-way ANOVA). All experiments were performed in triplicate and repeated twice for confirmation.

FIGS. 8A-8I. Drug screening for anti-SARS-CoV-2 constituents from Chinese herb medicines. (FIG. 8A) Vero E6 cells were cultured for 16 h, followed by infection with a clinical isolate of SARS-CoV-2 (HKU-001a) with a multiplicity of infection (MOI) of 0.01. All the compounds were treated at dosage of 10 μg/ml. (FIG. 8B and FIG. 8C) Chemical structure of 23-O-acetylalisol B (FIG. 8B) and 24-O-acetylalisol A (FIG. 8C). (FIG. 8D) Caco-2 cell line was infected with SARS-CoV-2 with MOI=0.01 and treated with 24-O-acetylalisol A in 4 dosages (10, 20, 30, 40 μM). Cell culture supernatant were collected for viral titer quantification by plaque assay at 48 h.p.i. (FIGS. 8E-8G) Cell viabilities of Caco-2 and VeroE6 were detected by MTT assay after 24 h and 48 h alisol B treatment. (FIG. 8H and FIG. 8I) SARS-CoV-2 pseudotyped particles pre-treated with camostat, and the relative mixture was culture with VeroE6 cells (FIG. 811) and VeroE6-TMPRSS2 cells (FIG. 8I). Data are presented as mean±S.E.M, *p<0.05, **p<0.01, ***p<0.001, relative to DMSO treated group, multiple comparison, dunnett test, one-way ANOVA. All experiments were performed in triplicate and repeated twice for confirmation.

FIGS. 9A-9D. Acute toxicity studies of alisol B in hamster for 14 days. Alisol B was intraperitoneal injected to hamster at one time (360 mg/kg). Organ samples and blood samples were collected on day 14 after the first administration. (FIG. 9A) Body weight changes in hamster. (FIG. 9B) ALT and AST activity in plasma. (FIG. 9C) Organ index. (FIG. 9D) Histological examination on heart, kidney, spleen, lung and liver morphology. Data shown as mean±S.E.M. of n=5 animals per group.

FIGS. 10A-10D. Alisol B decreases peroxynitrite (ONOO⁻) and reactive oxygen species (ROS) in vivo. (FIG. 10A) Representative fluorescence micrographs of HKYellow stained lung tissues of alisol B and vehicle treated SARS-CoV-2 infected hamster. (FIG. 10B) HKYellow fluorescence intensity (ONOO⁻) levels in lung tissues. (FIG. 10C) Representative fluorescence micrographs of hydroethidine (HET) stained lung tissues of alisol B and vehicle treated SARS-CoV-2 infected hamster. (FIG. 10D) HET fluorescence intensity (ROS) levels in lung tissues. All the data are presented as mean±S.E.M (*p<0.05, **p<0.01, ***p<0.001, relative to vehicle group, dunnett test, one-way ANOVA). All the quantification were conducted based on 2 views in one sample.

FIGS. 11A-11N. Immune suppression of alisol B in mice lymphocytes. (FIGS. 11A-11C and FIGS. 11E-11G) Representative flow cytometry results of Tfh cells (FIGS. 11A-11C) and Treg cells (FIGS. 11E-11G) treated with alisol B and DMSO as control. (FIG. 11D and FIG. 11H) Quantification of the percentage of Tfh cells (FIG. 11D) and Treg cells (FIG. 11H) positive cells in alisol B treated T cells. (FIGS. 11I-11M and FIG. 11N) M2 polarization results of alisol B treated macrophages. All the data are presented as mean±S.E.M (*p<0.05, **p<0.01, ***p<0.001, relative to vehicle group, Dunnett test, one-way ANOVA). All experiments were performed in triplicate and repeated twice for confirmation.

### BRIEF DESCRIPTION OF THE SEQUENCES

SEQ ID NO: 1 Forward primer targeting the RNA-dependent RNA polymerase/helicase (RdRP/Hel) gene region of SARS-CoV-2

SEQ ID NO: 2: Reverse primer targeting the RNA-dependent RNA polymerase/helicase (RdRP/Hel) gene region of SARS-CoV-2

SEQ ID NO: 3: Specific probe targeting the RNA-dependent RNA polymerase/helicase (RdRP/Hel) gene region of SARS-CoV-2

SEQ ID NO: 4: Forward primer targeting the MERS-CoV-NP

SEQ ID NO: 5: Reverse primer targeting MERS-CoV-NP

### DETAILED DISCLOSURE OF THE INVENTION

### Selected Definitions

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. The transitional terms/phrases (and any grammatical variations thereof) “comprising”, “comprises”, “comprise”, “consisting essentially of”, “consists essentially of”, “consisting” and “consists” can be used interchangeably.

The phrases “consisting essentially of” or “consists essentially of” indicate that the claim encompasses embodiments containing the specified materials or steps and those that do not materially affect the basic and novel characteristic(s) of the claim.

The term “about” means within an acceptable error range for the particular value as determined by one of ordinary skill in the art, which depends in part on how the value is measured, i.e., the limitations of the measurement system. In the context of compositions containing amounts of ingredients where the terms “about” is used, these compositions contain the stated amount of the ingredient with a variation (error range) of 0-10% around the value (X±10%). In other contexts the term “about” is provides a variation (error range) of 0-10% around a given value (X±10%). As is apparent, this variation represents a range that is up to 10% above or below a given value, for example, X±1%, X±2%, X±3%, X±4%, X±5%, X±6%, X±7%, X±8%, X±9%, or X±10%.

In the present disclosure, ranges are stated in shorthand to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. Values having at least two significant digits within a range are envisioned, for example, a range of 5-10 indicates all the values between 5.0 and 10.0 as well as between 5.00 and 10.00 including the terminal values. When ranges are used herein, combinations and subcombinations of ranges (e.g., subranges within the disclosed range) and specific embodiments therein are explicitly included.

As used herein, the term “subject” refers to an animal, needing or desiring delivery of the benefits provided by a drug. The animal may be for example, humans, pigs, horses, goats, cats, mice, rats, dogs, apes, fish, chimpanzees, orangutans, guinea pigs, hamsters, cows, sheep, birds, chickens, as well as any other vertebrate or invertebrate. These benefits can include, but are not limited to, the treatment of a health condition, disease or disorder; prevention of a health condition, disease or disorder; immune health; enhancement of the function of an organ, tissue, or system in the body. The preferred subject in the context of this invention is a human. The subject can be of any age or stage of development, including infant, toddler, adolescent, teenager, adult, or senior.

As used herein, the terms “therapeutically-effective amount,” “therapeutically-effective dose,” “effective amount,” and “effective dose” are used to refer to an amount or dose of a compound or composition that, when administered to a subject, is capable of treating, preventing, or improving a condition, disease, or disorder in a subject. In other words, when administered to a subject, the amount is “therapeutically effective.” The actual amount will vary depending on a number of factors including, but not limited to, the particular condition, disease, or disorder being treated, prevented, or improved; the severity of the condition; the weight, height, age, and health of the patient; and the route of administration.

As used herein, the term “treatment” refers to eradicating, reducing, ameliorating, or reversing a sign or symptom of a health condition, disease or disorder to any extent, and includes, but does not require, a complete cure of the condition, disease, or disorder. Treating can be curing, improving, or partially ameliorating a disorder. “Treatment” can also include improving or enhancing a condition or characteristic, for example, bringing the function of a particular system in the body to a heightened state of health or homeostasis.

As used herein, “preventing” a health condition, disease, or disorder refers to avoiding, delaying, forestalling, or minimizing the onset of a particular sign or symptom of the condition, disease, or disorder. Prevention can, but is not required, to be absolute or complete; meaning, the sign or symptom may still develop at a later time. Prevention can include reducing the severity of the onset of such a condition, disease, or disorder, and/or inhibiting the progression of the condition, disease, or disorder to a more severe condition, disease, or disorder.

In some embodiments of the invention, the method comprises administration of multiple doses of the compounds of the subject invention. The method may comprise administration of 1, 2, 3, 4, 5, 6, 7, 8, 9, 10, 15, 20, 25, 30, 35, 40, or more therapeutically effective doses of a composition comprising the compounds of the subject invention as described herein. In some embodiments, doses are administered over the course of 1 day, 2 days, 3 days, 4 days, 5 days, 6 days, 7 days, 10 days, 14 days, 21 days, 30 days, or more than 30 days. The frequency and duration of administration of multiple doses of the compositions is such as prevent or treat a viral infection. Moreover, treatment of a subject with a therapeutically effective amount of the compounds of the invention can include a single treatment or can include a series of treatments. It will also be appreciated that the effective dosage of a compound used for treatment may increase or decrease over the course of a particular treatment. Changes in dosage may result and become apparent from the results of testing for a virus. In some embodiments of the invention, the method comprises administration of the compounds at several time per day, including but not limiting to 2 times per day, 3 times per day, and 4 times per day.

As used herein, an “isolated” or “purified” compound is substantially free of other compounds. In certain embodiments, purified compounds are at least 60% by weight (dry weight) of the compound of interest. Preferably, the preparation is at least 75%, more preferably at least 90%, and most preferably at least 99%, by weight of the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

By “reduces” is meant a negative alteration of at least 1%, 5%, 10%, 25%, 50%, 75%, or 100%.

By “increases” is meant as a positive alteration of at least 1%, 5%, 10%, 25%, 50%, 75%, or 100%.

As used herein, a “pharmaceutical” refers to a compound manufactured for use as a medicinal and/or therapeutic drug.

### 23-O-Acetylalisol B (Alisol B)

The subject invention pertains to a method for treatment or prevention of a coronavirus infection or a symptom thereof, such as SARS-CoV-2, in a subject, comprising administering to the subject an effective amount of compounds that can target a coronavirus or a pharmaceutically acceptable salt, derivative, or prodrug thereof.

Alisol B may be administered to the human subject before or after initiation of the coronavirus infection, thereby treating the coronavirus infection. In some embodiments, the subject has the disease COVID-19 at the time that Alisol B is administered.

In certain embodiments, Alisol B can be administered after the viral infection. Alisol B can limit or prevent complications or symptoms of the previous infection.

Another aspect of the invention concerns a method for inhibiting a human coronavirus infection in a human cell, comprising contacting a viral particle and/or infected cell with Alisol B, or a pharmaceutically acceptable salt, derivative, or prodrug thereof, before or after the viral particle infects a cell.

The human coronavirus may be any type or subgroup, including alpha, beta, gamma, and delta. In some embodiments of the aforementioned methods of the invention, the human coronavirus is selected from among SARS-CoV-2, SARS-CoV, and MERS-CoV. In some embodiments of the aforementioned methods of the invention, the human coronavirus is a common human coronavirus, such as type 229E, NL63, OC43, and HKU1.

Another aspect of the invention concerns a composition comprising Alisol B, or a pharmaceutically acceptable salt, derivative, or prodrug thereof.

In one embodiment of the compositions and methods of the invention, Alisol B comprises one or more compounds disclosed herein and/or in Formula (I), or a structural or functional derivative thereof that retains activity inhibitory to a coronavirus infection, or a pharmaceutically acceptable salt of any of the foregoing.

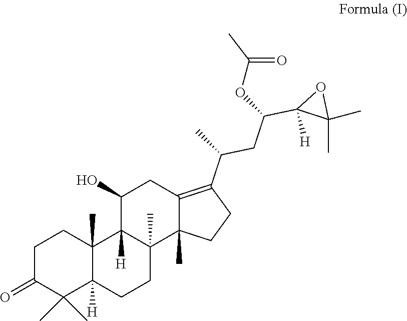

### Compositions and Treatment

Alisol B of the present invention can be formulated into pharmaceutically acceptable salt forms or hydrate forms. Pharmaceutically acceptable salt forms include the acid addition salts and include hydrochloric, hydrobromic, nitric, phosphoric, carbonic, sulfuric, and organic acids like acetic, propionic, benzoic, succinic, fumaric, mandelic, oxalic, citric, tartaric, maleic, and the like. Pharmaceutically acceptable base addition salts include sodium, potassium, calcium, ammonium, and magnesium salts. Pharmaceutically acceptable salts of the polypeptides of the invention can be prepared using conventional techniques.

Administration of Alisol B can be carried out in the form of an oral tablet, capsule, or liquid formulation containing a therapeutically effective amount of the active ingredient (Alisol B). Administration is not limited to oral delivery and includes intravascular (e.g., intravenous), intramuscular, or another means known in the pharmaceutical art for administration of active pharmaceutical ingredients.

Therapeutic or prophylactic application of Alisol B and compositions containing thereof, can be accomplished by any suitable therapeutic or prophylactic method and technique presently or prospectively known to those skilled in the art. Alisol B can be administered by any suitable route known in the art including, for example, oral, intramuscular, intraspinal, intracranial, nasal, rectal, parenteral, subcutaneous, or intravascular (e.g., intravenous) routes of administration. Administration of Alisol B can be continuous or at distinct intervals as can be readily determined by a person skilled in the art.

In some embodiments, an amount of Alisol B can be administered 1, 2, 3, 4, or times per day, for 1, 2, 3, 4, 5, 6, 7, or more days. Treatment can continue as needed, e.g., for several weeks. Optionally, the treatment regimen can include a loading dose, with one or more daily maintenance doses.

Alisol B and compositions comprising said Alisol B can be formulated according to known methods for preparing pharmaceutically useful compositions. Formulations are described in detail in a number of sources which are well known and readily available to those skilled in the art. For example, Remington's Pharmaceutical Science by E.W. Martin describes formulations which can be used in connection with the subject invention. In general, the compositions of the subject invention will be formulated such that an effective amount of Alisol B is combined with a suitable carrier in order to facilitate effective administration of the composition. The compositions used in the present methods can also be in a variety of forms. These include, for example, solid, semi-solid, and liquid dosage forms, such as tablets, pills, powders, liquid solutions or suspension, suppositories, injectable and infusible solutions, and sprays. The preferred form depends on the intended mode of administration and therapeutic application. The compositions also preferably include conventional pharmaceutically acceptable carriers and diluents which are known to those skilled in the art. Examples of carriers or diluents for use with Alisol B include, but are not limited to, water, saline, oils including mineral oil, ethanol, dimethyl sulfoxide, gelatin, cyclodextrans, magnesium stearate, dextrose, cellulose, sugars, calcium carbonate, glycerol, alumina, starch, and equivalent carriers and diluents, or mixtures of any of these. Formulations of Alisol B can also comprise suspension agents, protectants, lubricants, buffers, preservatives, and stabilizers. To provide for the administration of such dosages for the desired therapeutic treatment, pharmaceutical compositions of the invention will advantageously comprise between about 0.1% and 45%, and especially, 1 and 15% by weight of the total of Alisol B based on the weight of the total composition including carrier or diluent.

Alisol B can also be administered utilizing liposome technology, slow release capsules, implantable pumps, and biodegradable containers. These delivery methods can, advantageously, provide a uniform dosage over an extended period of time.

The subject invention also concerns a packaged dosage formulation comprising in one or more packages, packets, or containers Alisol B and/or composition of the subject invention formulated in a pharmaceutically acceptable dosage. The package can contain discrete quantities of the dosage formulation, such as tablet, capsules, lozenge, and powders. The quantity of Alisol B a dosage formulation and that can be administered to a patient can vary from about 1 mg to about 2000 mg, or about 1 mg to about 500 mg, or more typically about 5 mg to about 250 mg, or about 10 mg to about 100 mg. In some embodiments, the amount is in the range of 100 mg to 600 mg, to be administered 1, 2, 3, or 4 times per day, for 2, 3, 4, 5, 6, 7 or more days.

The subject invention also concerns kits comprising in one or more containers Alisol B. A kit of the invention can also comprise one or more compounds, biological molecules, or drugs. In one embodiment, a kit of the invention comprises Alisol B.

Optionally, the methods further comprise, prior to administering Alisol B to the subject, identifying the subject as having a human coronavirus infection (human coronavirus, generally, or a specific strain of coronavirus, such as SARS-CoV-2), or not having a human coronavirus infection. If the subject is identified as having a human coronavirus infection, Alisol B can be administered to the human subject as therapy. If the human subject is identified as not having a human coronavirus infection, Alisol B can be withheld, or Alisol B can be administered as prophylaxis, or an alternative agent can be given. The identifying step may comprise assaying a biological sample (e.g., blood, saliva, or urine) obtained from the subject for the presence of human coronavirus nucleic acids or human coronavirus proteins, such as SARS-CoV-2 nucleic acids or proteins. In some embodiments, assaying includes the use of reverse transcriptase-polymerase chain reaction (RT-PCR), immunological assay (e.g., ELISA), or Plaque-reduction neutralization testing (PRNT).

Thus, optionally, the methods include, prior to administration of Alisol B, or re-administration of Alisol B, determining whether the subject has a human coronavirus infection or one or more symptoms consistent with a human coronavirus infection. Some individuals infected with coronavirus will not know they have the infection because they will not have symptoms.

In some embodiments of the methods of the invention, the human coronavirus is selected from among SARS-CoV-2, SARS-CoV, and MERS-CoV. SARS-CoV-2 is a novel human coronavirus that causes coronavirus disease 2019, also known as COVID-19 and COVID19. MERS-CoV is the beta coronavirus that causes Middle East Respiratory Syndrome, or MERS. SARS-CoV is the beta coronavirus that causes severe acute respiratory syndrome, or SARS.

In some embodiments of the methods of the invention, the human coronavirus is a common human coronavirus, such as type 229E (an alpha coronavirus), NL63 (an alpha coronavirus), 0C43 (a beta coronavirus), and HKU1 (a beta coronavirus).

The symptoms of a coronavirus infection depend on the type of coronavirus and severity of the infection. If a subject has a mild to moderate upper-respiratory infection, such as the common cold, symptoms may include: runny nose, headache, cough, sore throat, fever, and general feeling of being unwell. Some coronaviruses can cause severe symptoms. These infections may turn into bronchitis and pneumonia, which can cause symptoms such as fever (which can be quite high with pneumonia), cough with mucus, shortness of breath, and chest pain or tightness when the subject breaths or coughs.

The clinical spectrum of SARS-CoV-2 may range from mild disease with non-specific signs and symptoms of acute respiratory illness, to severe pneumonia with respiratory failure and septic shock. Asymptomatic infections have also been reported.

To diagnose coronavirus infections, healthcare providers typically take the subject's medical history and ask the subject their symptoms, do a physical examination, and may conduct laboratory tests on a biological sample such as blood, or a respiratory specimen such as sputum or a throat swab.

SARS-CoV-2 RNA has been detected from upper and lower respiratory tract specimens, and the virus has been isolated from upper respiratory tract specimens and bronchoalveolar lavage fluid. SARS-CoV-2 RNA has been detected in blood and stool specimens. The duration of SARS-CoV-2 RNA detection in the upper and lower respiratory tracts and in extrapulmonary specimens has not been determined. It is possible that RNA could be detected for weeks, which has occurred in some cases of MERS-CoV or SARS-CoV infection. Viable SARS-CoV has been isolated from respiratory, blood, urine, and stool specimens, and viable MERS-CoV has been isolated from respiratory tract specimens.

Treatment methods optionally include steps of advising that the subject get plenty of rest and drink fluids for hydration and administration of agents that alleviate symptoms of coronavirus infection, such as those that reduce fever and pain (e.g., acetaminophen and/or paracetamol), particularly for common human coronavirus infections. The methods may include administration of the fluids to the subject for hydration.

The subject may be any age or gender. In some cases, the subject may be an infant or older adult. In some embodiments, the subject is 40 years of age or older. In some embodiments, the subject is 55 years of age or older. In some embodiments, the subject is 60 years of age or older. In some embodiments, the subject is an infant. In some embodiments, the subject (of any age or gender) has heart or lung disease, diabetes, or a weakened immune system.

The invention further provides kits, including Alisol B and pharmaceutical formulations, packaged into suitable packaging material, optionally in combination with instructions for using the kit components, e.g., instructions for performing a method of the invention. In one embodiment, a kit includes an amount of Alisol B and instructions for administering Alisol B to a subject in need of treatment on a label or packaging insert. In further embodiments, a kit includes an article of manufacture, for delivering Alisol B into a subject locally, regionally or systemically, for example.

As used herein, the term “packaging material” refers to a physical structure housing the components of the kit. The packaging material can maintain the components sterilely, and can be made of material commonly used for such purposes (e.g., paper, corrugated fiber, glass, plastic, foil, ampules, etc.). The label or packaging insert can include appropriate written instructions, for example, practicing a method of the invention, e.g., treating a human coronavirus infection, an assay for identifying a subject having a human coronavirus infection, etc. Thus, in additional embodiments, a kit includes a label or packaging insert including instructions for practicing a method of the invention in solution, in vitro, in vivo, or ex vivo.

Instructions can therefore include instructions for practicing any of the methods of the invention described herein. For example, pharmaceutical compositions can be included in a container, pack, or dispenser together with instructions for administration to a subject to treat a human coronavirus infection. Instructions may additionally include appropriate administration route, dosage information, indications of a satisfactory clinical endpoint or any adverse symptoms that may occur, storage information, expiration date, or any information required by regulatory agencies such as the Food and Drug Administration or European Medicines Agency for use in a human subject.

The instructions may be on “printed matter,” e.g., on paper or cardboard within the kit, on a label affixed to the kit or packaging material, or attached to a vial or tube containing a component of the kit. Instructions may comprise voice or video tape and additionally be included on a computer readable medium, such as a disk (floppy diskette or hard disk), optical CD such as CD- or DVD-ROM/RAM, magnetic tape, electrical storage media such as RAM and ROM and hybrids of these such as magnetic/optical storage media.

Kits can additionally include a buffering agent, a preservative, or an agent for stabilizing Alisol B. The kit can also include control components for assaying for the presence of human coronavirus, e.g., a control sample or a standard. Each component of the kit can be enclosed within an individual container or in a mixture and all of the various containers can be within single or multiple packages.

In certain embodiments, 23-O-Acetylalisol B can broadly and dose-dependently inhibit coronavirus (CoVs), including MERS-CoV, SARS-CoV-2, SARS-CoV-2 Alpha and Delta variants in vitro and in vivo. 23-O-Acetylalisol B can have anti-inflammation and immunomodulation effects and can have therapeutic effects to significantly ameliorate the CoVs infection-induced lung damage. 23-O-Acetylalisol B can treat severe acute respiratory syndrome (SARS) by broadly inhibiting CoVs and immunomodulation. In addition, based on the effects on the human lymphocytes (T cells, B cells and macrophages), 23-O-Acetylalisol B also can be an immunomodulation agent for immune disorders.

In certain embodiments, 23-O-Acetylalisol B can, broadly and dose-dependently, reduce viral replication in cells infected with different CoVs species, including, for example, MERS-CoV, SARS-CoV-2, SARS-CoV-2 Alpha and Delta variants.

In certain embodiments, 23-O-Acetylalisol B can exhibit strong antiviral activity by reducing the replication of a coronavirus, including, for example, SARS-CoV-2 and SARS-CoV-2 Delta variants in lung tissues.

In certain embodiments, 23-O-Acetylalisol B can have immunomodulation effects against a cornovirus, including, for example, SARS-CoV-2 and SARS-CoV-2 Delta variants induced lung injury via inhibiting the infiltrations of CD11b-positive macrophages and CD3-positive T cells into the lung tissues.

In certain embodiments, 23-O-Acetylalisol B can inhibit inflammation and oxidative stress by decreasing reactive oxygen species (ROS) and reactive nitrogen species (RNS) in lung tissues infected with a coronavirus, including, for example, SARS-CoV-2.

In certain embodiments, 23-O-Acetylalisol B can modulate the immune responses through increasing IgM B cell populations for humoral immunity in the lung tissues after infected by a coronavirus, including, for example, SARS-CoV-2 and SARS-CoV-2 Delta variants. Furthermore, 23-O-Acetylalisol B can promote the proliferation and differentiation of human B cells. Those results represent the enhancement of immune defense capacity against the infections of SARS-CoV-2 and SARS-CoV-2 Delta variants.

In certain embodiments, 23-O-Acetylalisol B can inhibit the proliferation of human T lymphocytes and macrophages. Accordingly, the present invention provides a novel antiviral drug 23-O-Acetylalisol B for treatment of CoVs infective diseases. The immunomodulation and anti-inflammation bioactivities can be also used for treating autoimmune disorders, such as, for example, multiple sclerosis, systemic lupus erythematosus, and rheumatoid arthritis treatment. In certain embodiments, the autoimmune disorders can be partly induced by IL-17, IFN-γ, IL-6 and IP10 (CXCL10). In certain embodiments, 23-O-Acetylalisol B can inhibit the proliferation of human T lymphocytes and macrophages, which can act as an immunosuppressive agent to treat autoimmune disorders. In certain embodiments, 23-O-Acetylalisol B can reduce the amount, concentration, or content of IL-17, IFN-γ, IL6, and IP10 (CXCL10) secretion.

### Materials and Methods

### Chemicals, Cells and Virus

23-O-Acetylalisol B with purity ≥98% was purchased from Chengdu Push Bio-technology Co., Ltd., China. Human colon Caco-2 cells (ATCC, HTB-37, Manassas, Va.) and monkey Vero E6 cells (ATCC, CRL-1586) were applied for antiviral studies which are highly sensitivity to each CoV replication, correspondingly. Cells were maintained in high glucose Dulbecco's modified Eagle medium (DMEM; Gibco, Thermo Fisher, Waltham, Mass.) supplemented with 10% fetal bovine serum (FBS; Gibco), 1% penicillin/streptomycin (PS; Gibco). The SARS-CoV-2 HKU-001a strain (GenBank accession number: MT230904) was isolated from the nasopharyngeal aspirate specimen of a laboratory-confirmed COVID-19 patient in Hong Kong [13]. The SARS-CoV-2 Isolate USA-WA1/2020 was deposited by the Centers for Disease Control and Prevention and obtained through BEI Resources. The MERS-CoV (HCoV-EMC/2012) was a gift from Dr. Ron Fouchier. The SARS-CoV-2 B.1.1.7 lineage (Alpha variant) and B.1.617.2 lineage (Delta variant) were archived in Department of Microbiology, The University of Hong Kong. All experiments involving live SARS-CoV-2, SARS-CoV-2 Alpha variant, SARS-CoV-2 Delta variant and MERS-CoV followed the approved standard operating procedures of the Biosafety Level 3 facility at the University of Hong Kong we previously described.

### Antiviral Evaluation In Vitro

Caco-2 cells and VeroE6 cells were infected with SARS-CoV-2 HKU-001a, SARS-CoV-2 alpha variant (B.1.1.7), SARS-CoV-2 delta variant (B.1.617.2) and MERS-CoV with 0.1 multiplicity of infection (MOI). After two hours infection, the inoculum was removed, and the cells were washed 3 times with PBS. The infected cells were culture in DMEM medium with 2 mM HEPES (Gibco), 1× GlutaMAX (Gibco), 100 U/mL penicillin, 100 μg/mL streptomycin, 20 μg/mL vancomycin, 20 μg/mL ciprofloxacin, 50 μg/mL amikacin, and 50 μg/mL nystatin. Supernatants and cell lysis were collected at 24 hours post inoculation (hpi) for qRT-PCR assays. Real-time one-step qRT-PCR was used for quantitation of SARS-CoV-2 and SARS-CoV-2 Delta variant viral load using the QuantiNova Probe RT-PCR kit (Qiagen, Hilden, Germany) with a LightCycler 480 Real-Time PCR System (Roche, Basel, Switzerland). Each 20 μl reaction mixture contained 10 μl of 2×QuantiNova Probe RT-PCR Master Mix, 1.2 μl of RNase-free water, 0.2 μl of QuantiNova Probe RT-Mix, 1.6 μl each of 10 μM forward and reverse primer, 0.4 μl of 10 μM probe and 5 μl of extracted RNA as the template. Reactions were incubated at 45° C. for 10 min for reverse transcription, 95° C. for 5 min for denaturation, followed by 45 cycles of 95° C. for 5 s and 55° C. for 30 s. Signal detection was carried out and measurements were made in each cycle after the annealing step. The cycling profile ended with a cooling step at 40° C. for 30 s. The primers and probe sequences were against the RNA-dependent RNA polymerase/helicase (RdRP/Hel) gene region of SARS-CoV-2 with the Forward primer: 5′-CGCATACAGTCTTRCAGGCT-3′ (SEQ ID NO: 1); Reverse primer: 5′-GTGTGATGTTGAWATGACATGGTC-3′ (SEQ ID NO: 2); specific probe: 5′-FAM TTAAGATGTGGTGCTTGCATACGTAGAC-IABkFQ-3′ (SEQ ID NO: 3). MERS-CoV: MERS-CoV-NP-F CAAAACCTTCCCTAAGAAGGAAAAG (SEQ ID NO: 4), and MERS-CoV-NP-R GCTCCTTTGGAGGTTCAGACAT (SEQ ID NO: 5).

### Cell Viability Assay

Cell viability of VeroE6 and Caco-2 was tested by 3-(4,5 dimethylthiazol-2-yl)-2,5-diphenyltetrazolium bromide (MTT) assay. Briefly, after 24 hours Alisol B treatment, the cells were incubated in medium contained with 0.5 mg/mL MTT for 4 h at 37° C. Then, the culture medium was removed, 150 μL DMSO was added into each well and mixed for 10 min. The absorbance was read by Multi-Plate Reader (Model 680, Bio-Rad, Hercules, Calif.) at 490 nm wavelength.

### Human Lymphocytes Culture

Human T cells, B cells and monocytes were isolated from PBMC of a healthy donor by magnetic-activated cell sorting (MACs) method. Anti-hCD3 and anti-hCD28 antibody were pre-coated for the activation and expansion of human T cells. T cell proliferation was assessed by staining T cells with a fluorescent tracking dye, carboxyfluorescein succinimidyl ester (CFSE) before starting the culture. Th17 cells were induced by TGFβ/IL-6/ANTI-IFNγ for 72 h. After 72 h, cells were resuspended to analysis cell proliferation by flow cytometry (Agilent, Quanteon, Santa Clara, Calif.). Culture medium was collected to detect the release of th17 within 3 days by Elisa kit (Biolegend, San Diego, Calif.).

### Endogenous ROS and Peroxynitrite Staining

In vivo peroxynitrite and ROS levels were determined using the ONOO⁻ sensitive HKYellow dye (20 μM) [18] and ROS-sensitive hydroethidine dye (HKT, 20 μM; Invitrogen, Waltham, Mass.). Fresh lung tissue (˜1 cm²) was collected at 4 days Alisol B treatment. Lung tissues were stained HKYellow and HKT solution 2 hours prior to fixation. After staining, fresh lung samples were sectioned into 20 μm cryosection slices for imaging. Fluorescent intensity of lung tissues was analyzed using ImageJ software.

### Antiviral Evaluation in a SARS-CoV-2 and SARS-CoV-2 Delta Variant Infected Hamster Model

Male Syrian hamsters, aged 6-10 weeks old, were obtained from the Chinese University of Hong Kong Laboratory Animal Service Centre through the HKU Centre for Comparative Medicine Research. The hamsters were kept in biosafety level 2 housing and given access to standard pellet feed and water ad libitum as previously described [13]. All animal care and experimental procedures were approved by the University Committee on the Use of Live Animals in Teaching and Research in the University of Hong Kong (Reference code: CULATR no. 5838-21). Experimentally, each hamster was intranasally inoculated with 10⁴PFU of SARS-CoV-2 and SARS-CoV-2 delta variant in 100 μL PBS under intraperitoneal ketamine (200 mg/kg) and xylazine (10 mg/kg) anesthesia.

The dosage of Alisol B for hamster treatment was 60 mg/kg/day based on the toxicity studies. Acute toxicity study of Alisol B was choice at 360 mg/kg and 420 mg/kg for male hamster. The solvent system was ethanol, PEG400 and saline (10:3:2) at 60 mg/mL due to the poor water solubility of Alisol B that used as vehicle. Specifically, 60 mg/kg (hamster)×0.13 (conversion factor)=7.8 mg/kg (human equivalent dose), and a 60 kg human requires 7.8×60 kg=468 mg Alisol B per day. For acute toxicity study, Alisol B was intraperitoneal injected to hamster at one time with 7 times than treatment dosage (420 mg/kg). Before injection, all the solutions were filtered by 0.22 μM filter. The body weight change and activity will be monitored daily and for 14 days. Therapeutic procedure of Alisol B treatment was applied intraperitoneal administration on 1, 2, 3 dpi (60 mg/kg) with first dosage given at 24 hpi. Animals were sacrificed at 4 dpi for virological and histopathological analyses. Viral yield in the lung tissue homogenates were detected by RT-qPCR methods. ELISA kit was used to detect the interferon gamma level in the hamster sera on 4 dpi according to the manufacture's recommendations (Bioscience). The lung tissue pathology of infected hamster was examined by H&E staining in accordance with an established protocol [19].

### Transcriptome Analysis

The quality of RNA samples of lung tissue for RNA-seq reads were checked by FastQC (v0.11.7) (see Worldwide website: bioinformatics.babraham.ac.uk/projects/fastqc/). Library construction was performed using Nextera XT kit following the manufacture's protocol. Reads with low quality regions and adapter contamination were removed by Cutadapt version 1.16 and only reads with length ≥30 were recognized as high-quality reads. The transcriptome alignment/mapping to each gene were done using TopHat version 2.1.1 with default parameters. All the samples had over 80% mapping with hamster reference MesAur1.0 (GCA_000349665.1) downloaded from Ensemble. Cut-off criteria for the low expression gene were filtered out with CPM threshold value of 1 using limma-voom. Read counts were normalized by Trimmed Mean of M-values method and differentially expressed genes were calculated using R package edgeR (v3.28.1). Genewise Negative Binomial Generalized Linear Models with Quasi-likelihood Tests (glmQLFit) method was used for statistical tests. The value of False Discovery Rate (FDR)≤0.05 was identified as the differential gene expression. The pathway analysis was performed by R package clusterProfiler42 (v3.14.3) and Metascape43. Heatmaps were plotted using R package pheatmap (v1.0.12) (Kolde, R. (2013). pheatmap: Pretty Heatmaps. R package version 0.7.7. see Worldwide website: CRAN.R-project.org/package=pheatmap). Other plots were generated by R package ggplot2 (v3.3.0) (Wickham H (2016). ggplot2: Elegant Graphics for Data Analysis. Springer-Verlag New York. ISBN 978-3-319-24277-4, see Worldwide website: ggplot2.tidyverse.org). PCA analysis was performed by R package factoextra (1.0.7).

### Immunofluorescence

The collected lung tissue was post-fixed with 4% PFA for 48 hours, completely dehydrated in 30% sucrose solution at 4° C. and embedded in O.C.T. The lung tissue was cut into 25 μm sections as frozen slices and stored at −20° C. For immunofluorescence imaging, the cells were cultured in Poly-D-Lysine coated 12 mm microscope slides (0111500; GmbH & Co. KG, Germany). Samples were processed with antigen-retrieved citrate acid buffer (pH 6.0) and microwave for 20 min. The samples were permeabilized and blocked with PBS containing 5% goat serum and 0.3% Triton X-100 for 1 hour at room temperature. After blocking, the samples were incubated with primary antibodies and stained with fluorochrome conjugated secondary antibodies, counterstained the nucleus with DAPI and mounted with antifade medium (Dako, Agilent). Cell images were obtained by regular confocal microscope (Zeiss LSM 800, Germany; Core facility in LSK Faculty of Medicine, HKU) and analyzed by Zeiss software. Specific primary antibodies included rabbit anti-CD3 (Abcam, 1:400, Cambridge, UK), rabbit anti-CD11b (Novus, 1:400, Centennial, Colo.), rabbit anti-IgM (Abnova, 1:400, Taipei City, Taipei, Taiwan) and rabbit antiserum against SARS-CoV-2-N protein.

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### Example 1—23-O-Acetylalisol B (Alisol B) has Antiviral Activity by Inhibiting Pan-Coronavirus Infection In Vivo

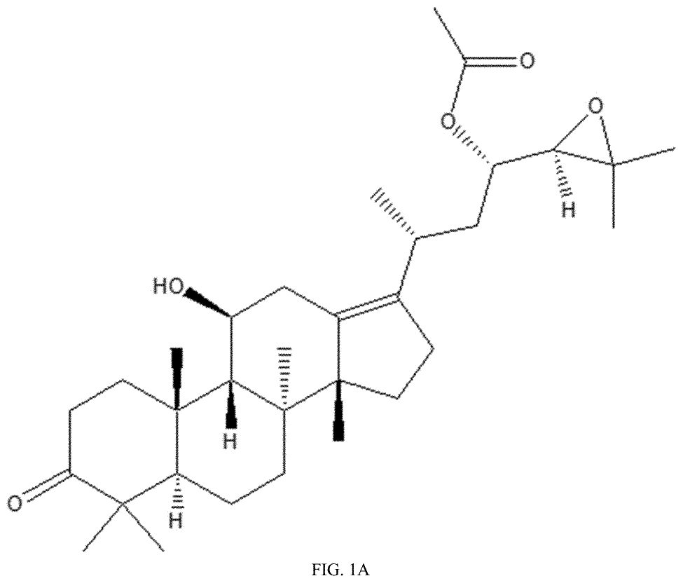

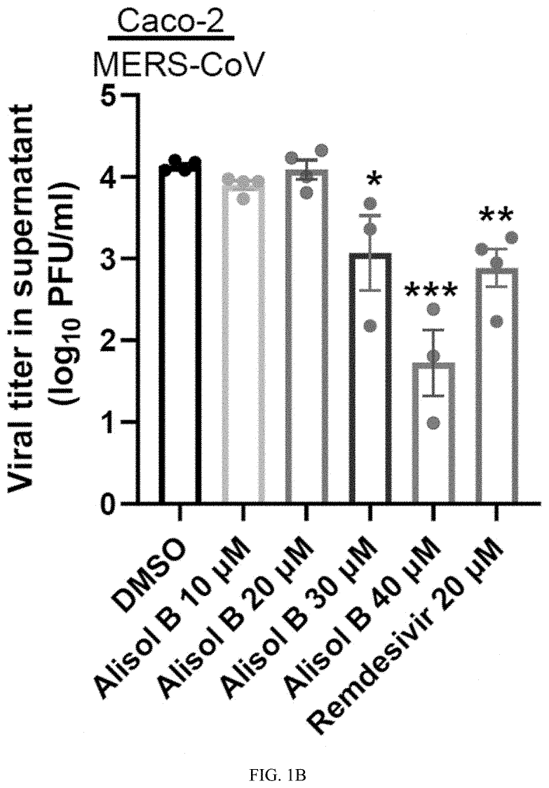

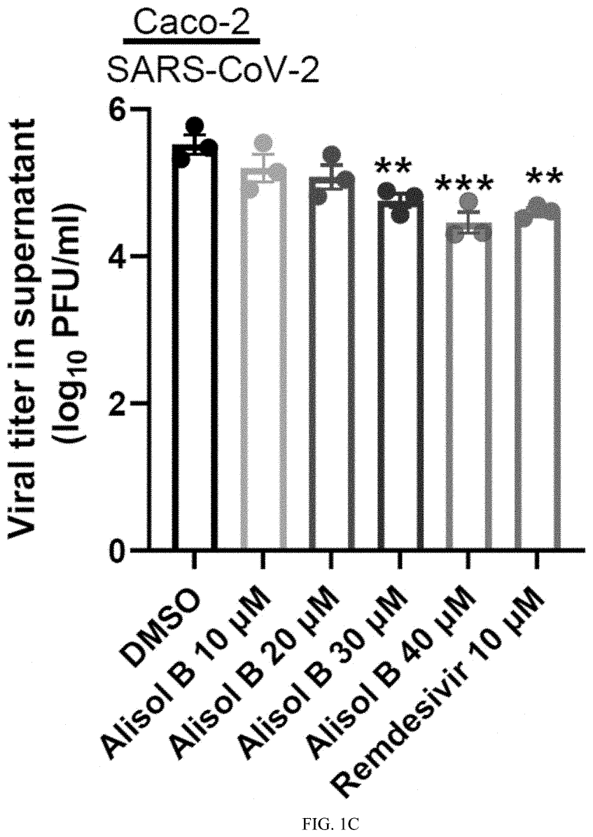

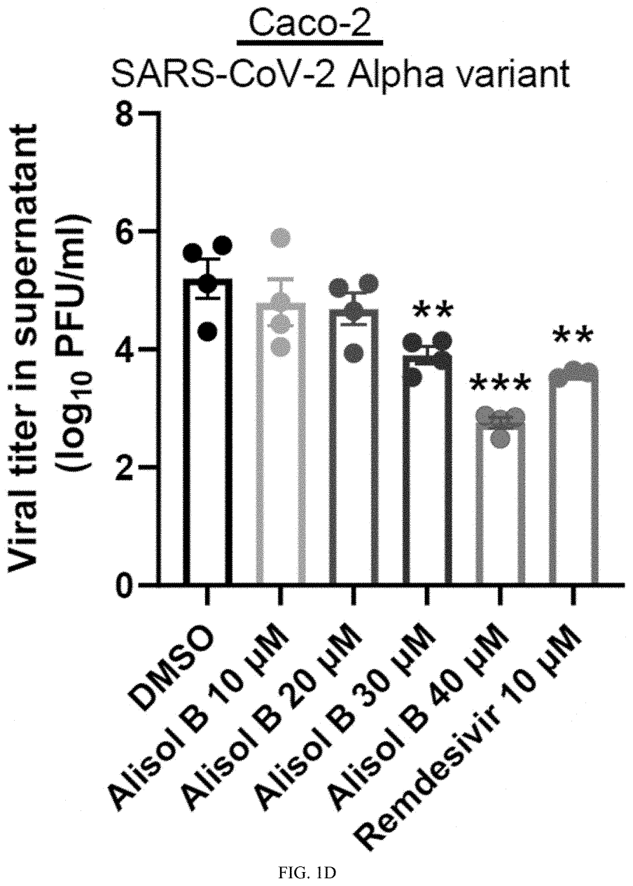

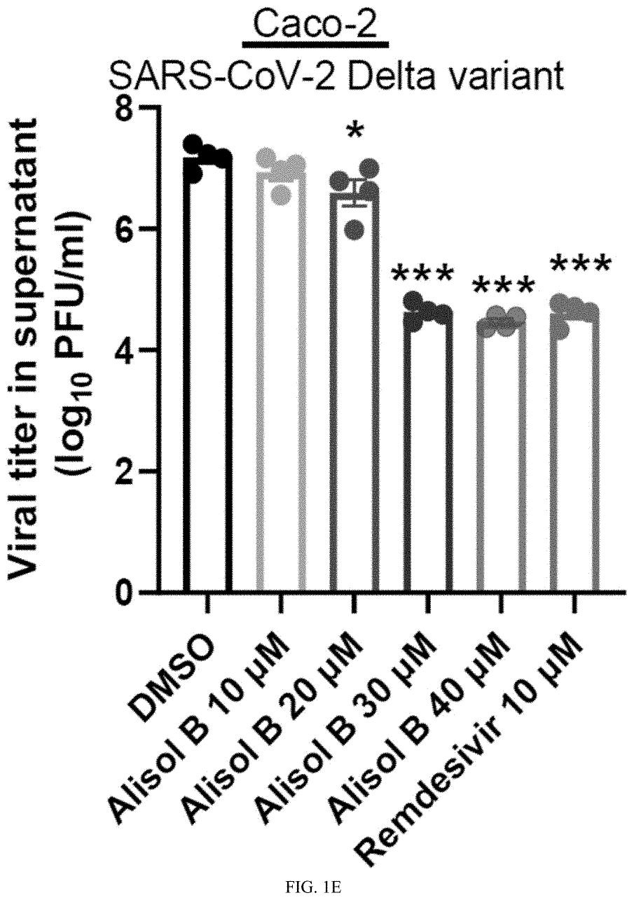

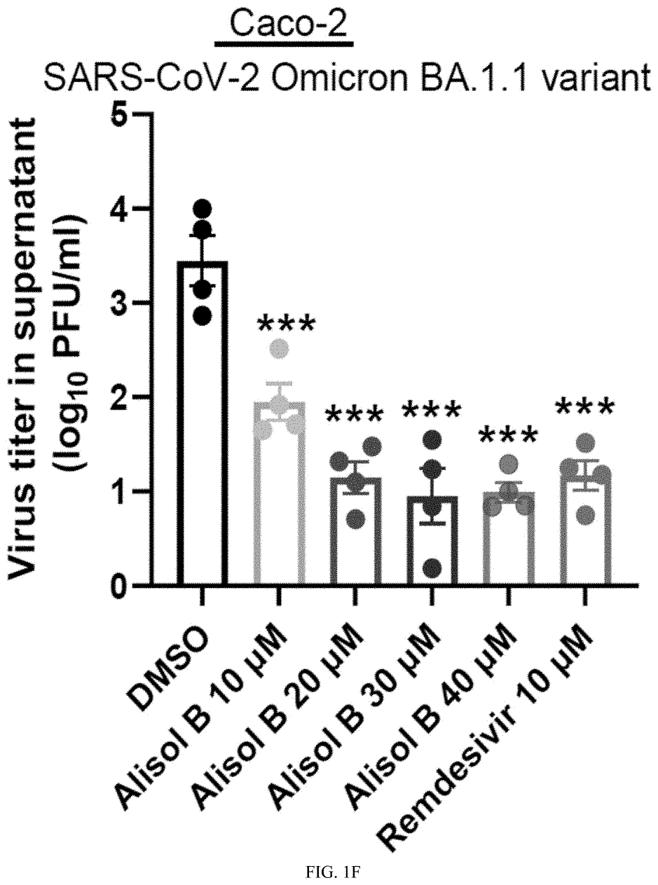

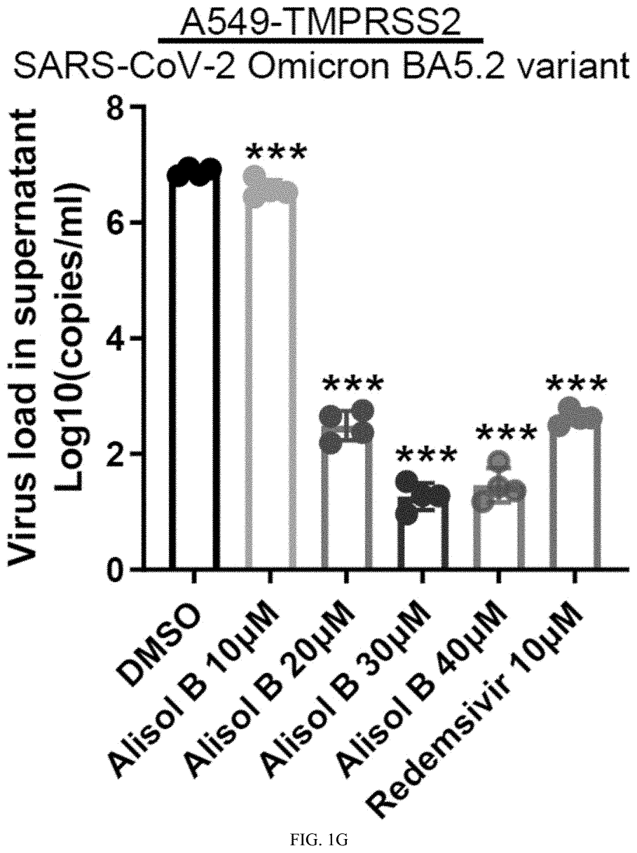

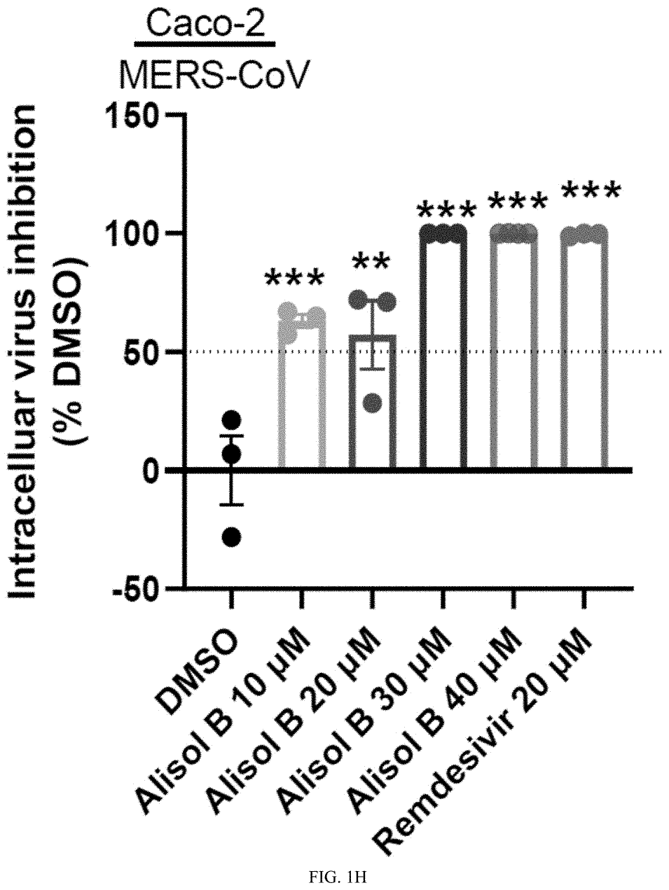

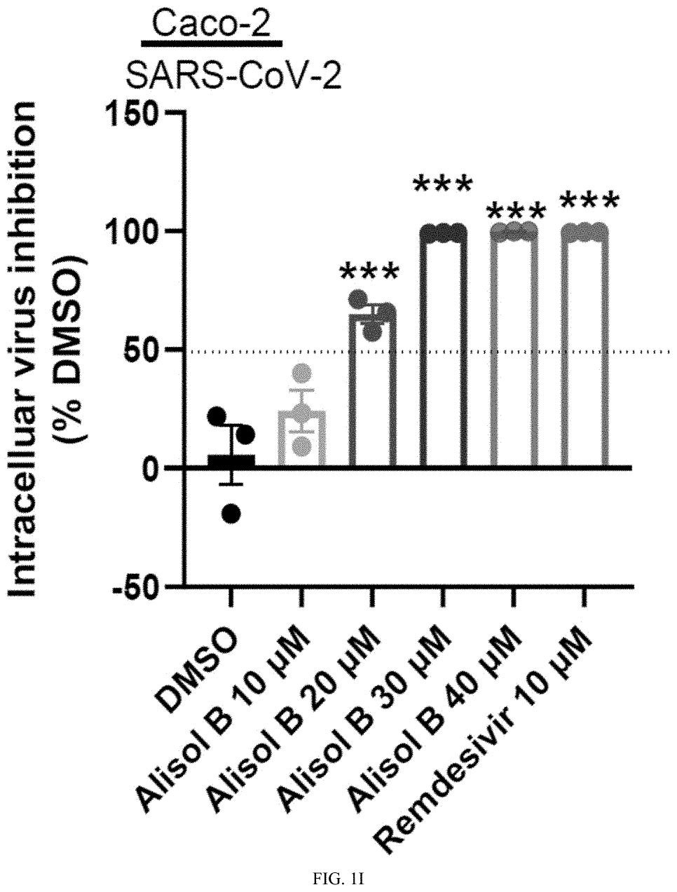

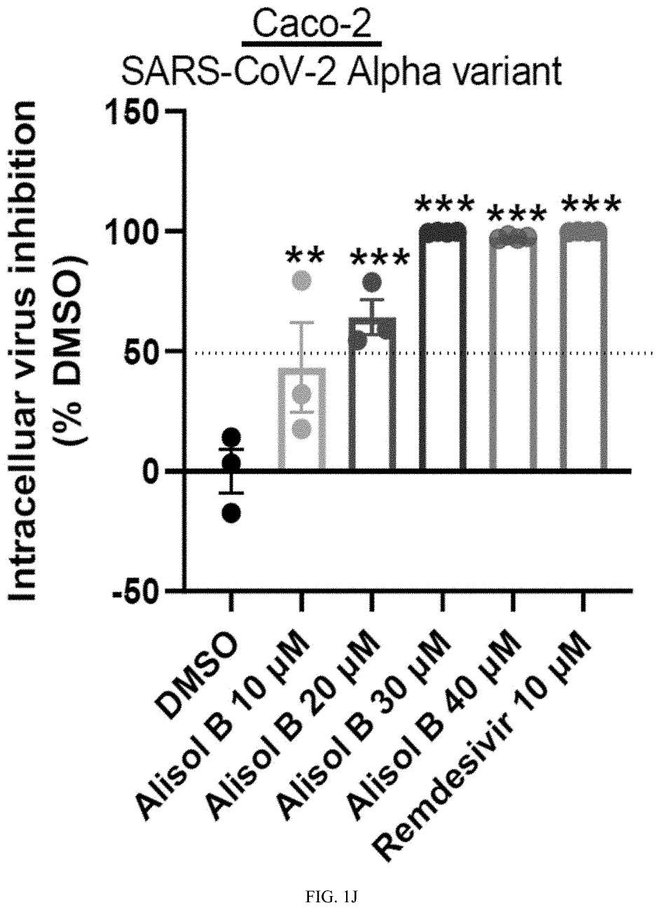

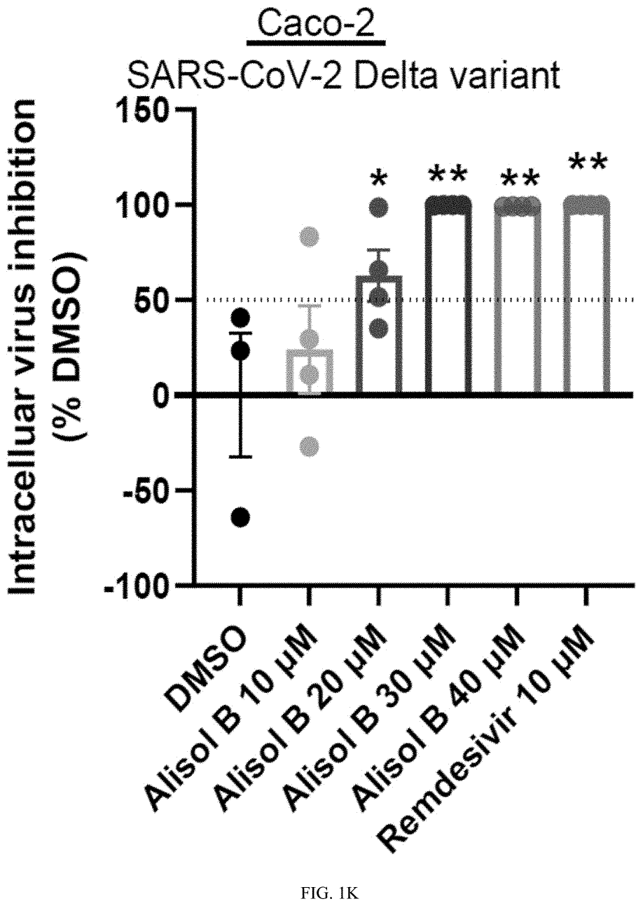

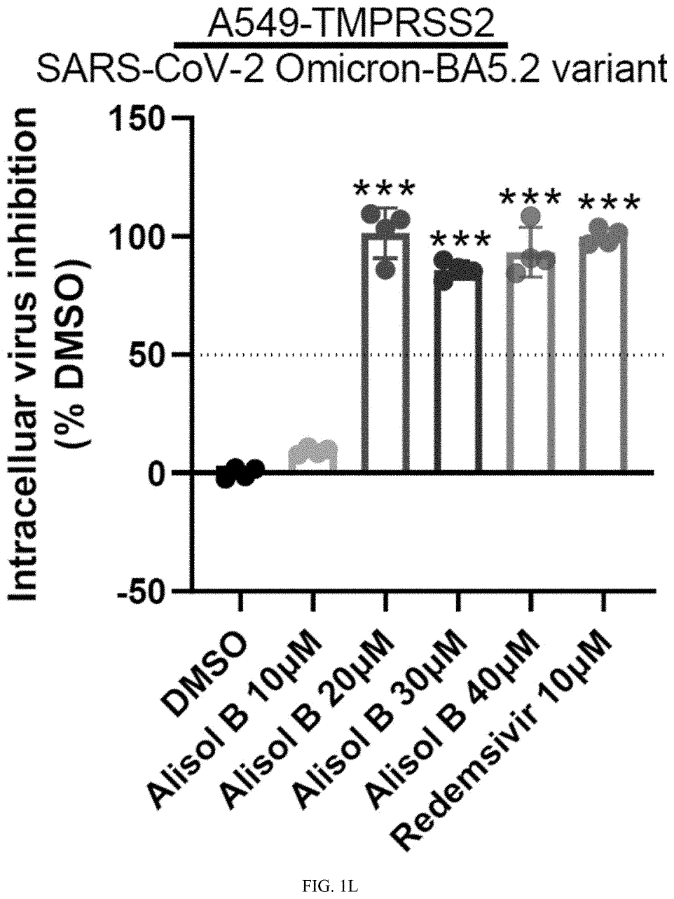

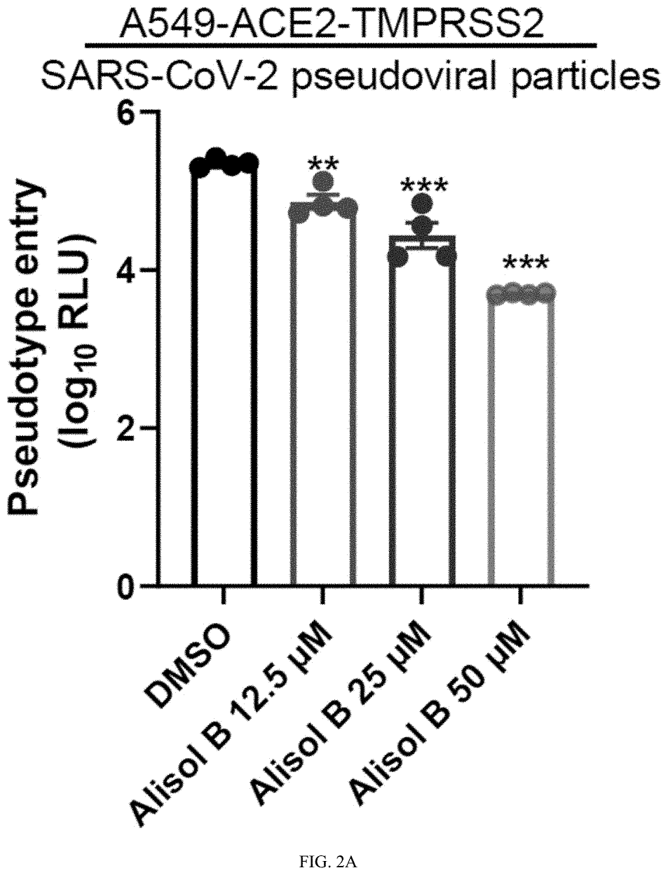

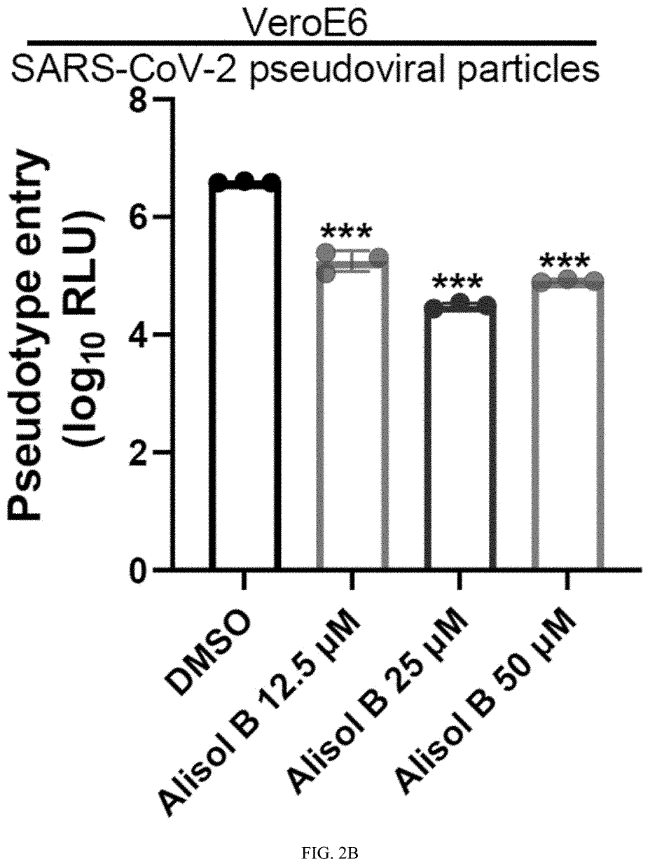

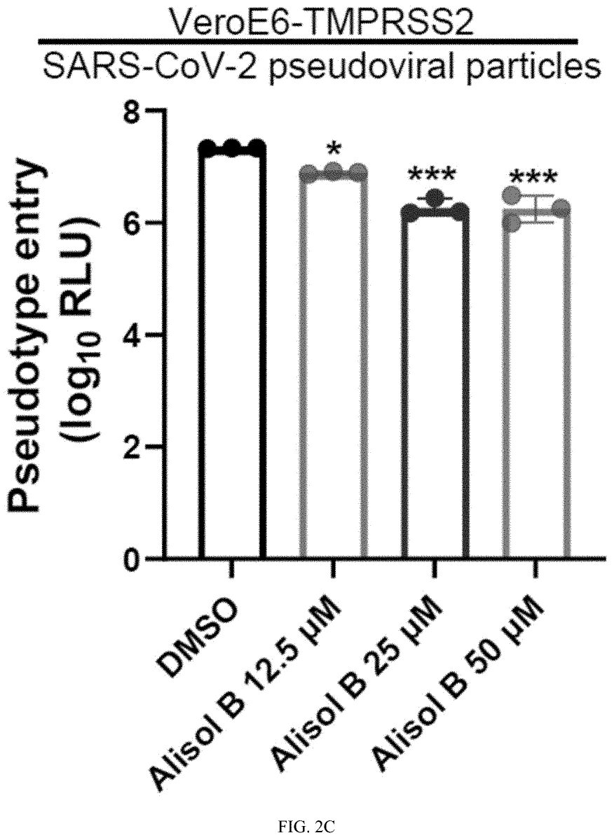

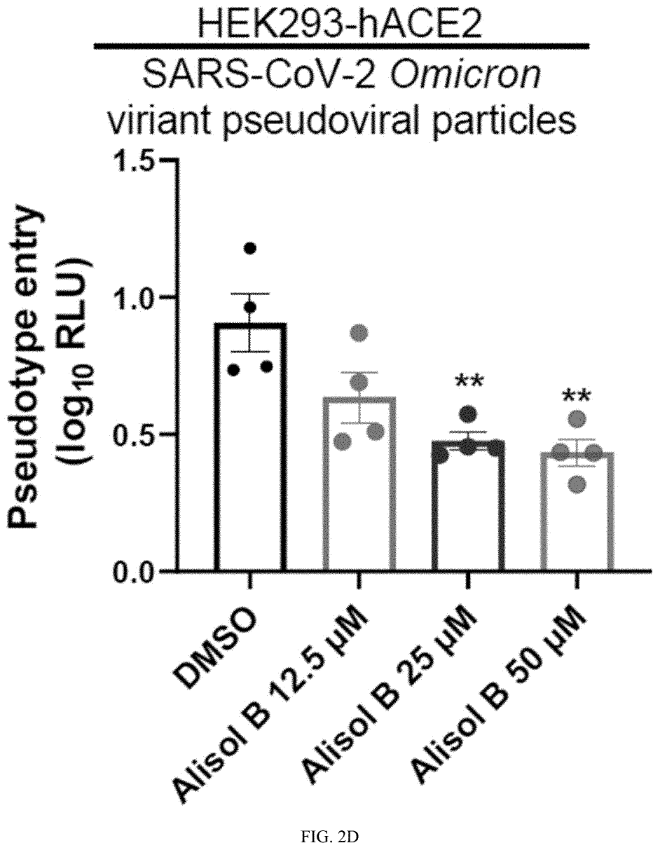

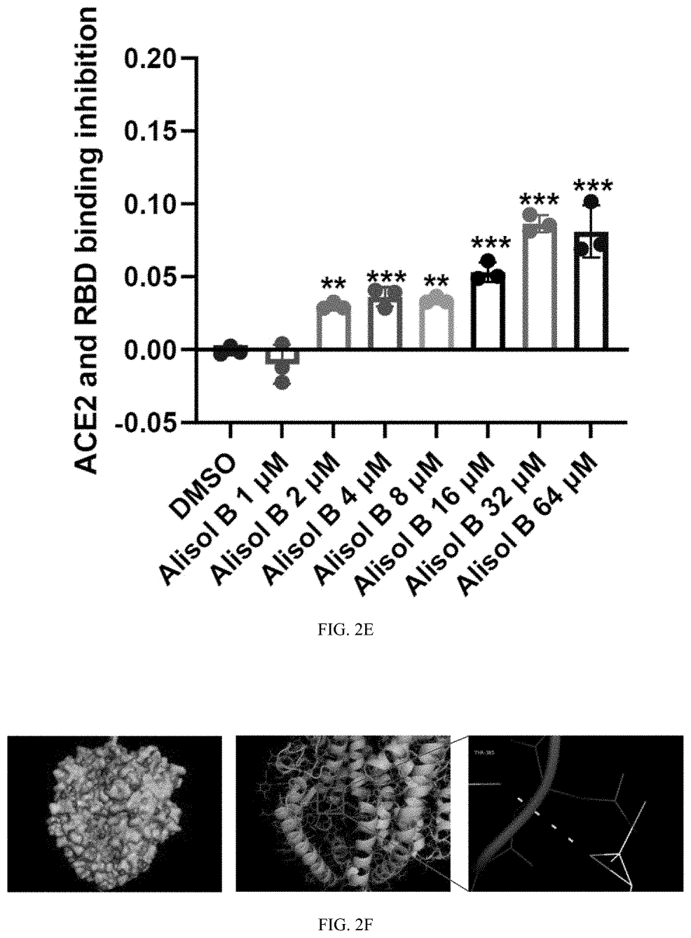

We performed in vitro cellular experiments by using VeroE6 cells and Caco-2 cells infected with different species of CoVs. The results revealed that Alisol B significantly inhibited SARS-CoV-2 replication in the VeroE6 cells. We then characterized the antiviral activity of Ali sol B in the Caco-2 cell line, which were reported to support CoVs replication [13, 14]. Alisol B treatment dose-dependently antagonized viral replication in the Caco-2 cells (FIGS. 1B and 1F; 1H-1K). To explore whether Alisol B confers cross-protection against other epidemic and mutated CoVs, we performed viral load reduction assays for MERS-CoV, SARS-CoV-2 alpha variant and SARS-CoV-2 delta variant. After treated with Alisol B, viral yields in the culture cell supernatants were significantly decreased when the Caco-2 cells were infected MERS-CoV, SARS-CoV-2 alpha variant and SARS-CoV-2 delta variants in a dose-dependent manner (FIGS. 1B-1F; 1H-1K).

### Example 2—23-O-Acetylalisol B has Antiviral Activity by Reducing the Replications of SARS-CoV-2 and SARS-CoV-2 Delta Variants in Lung Tissues of Hamster Covid-19 Model

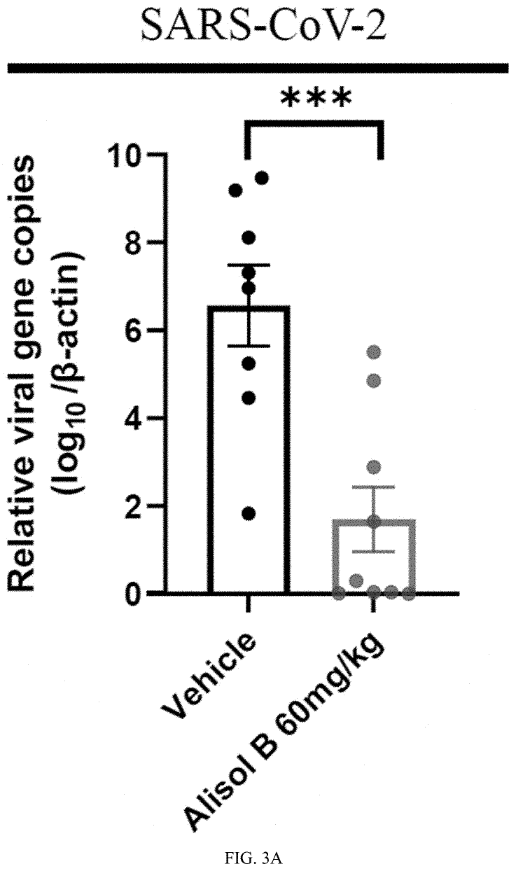

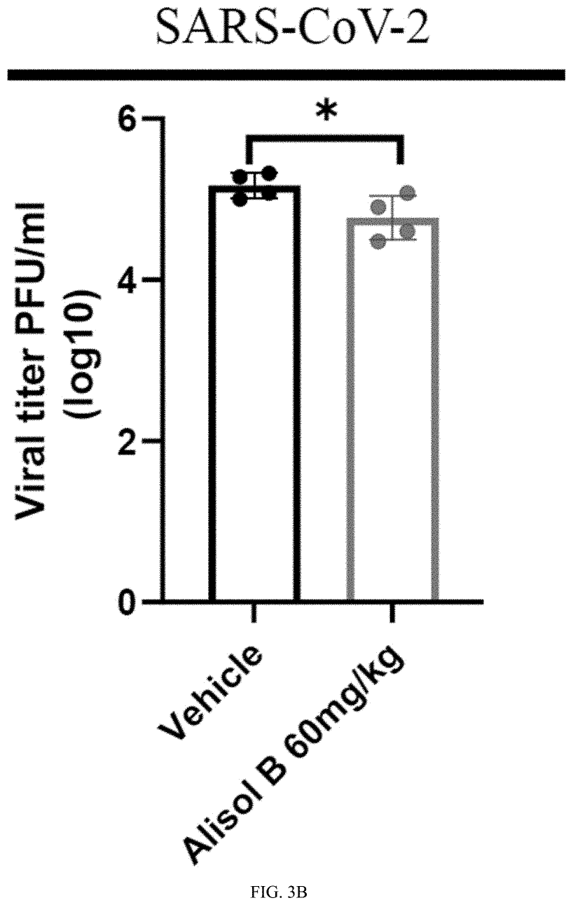

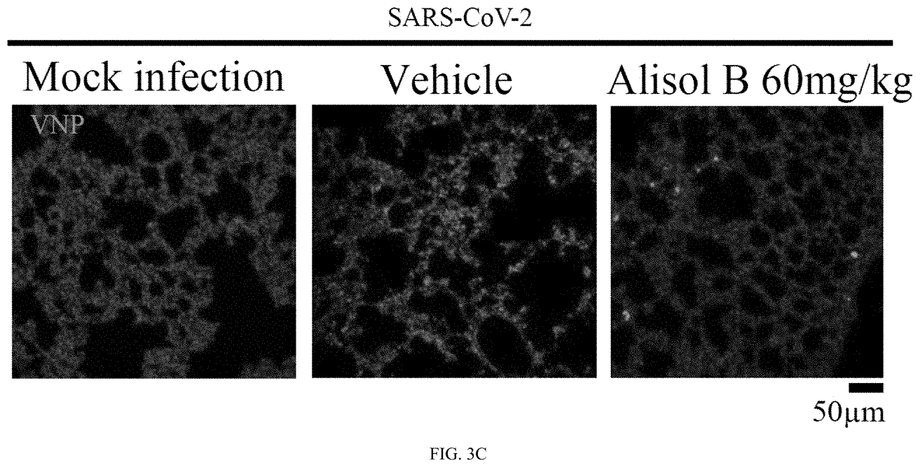

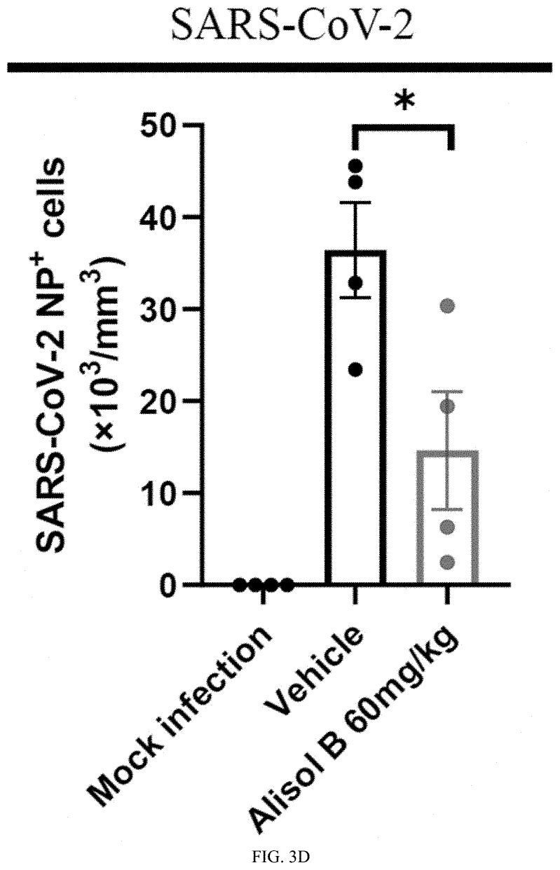

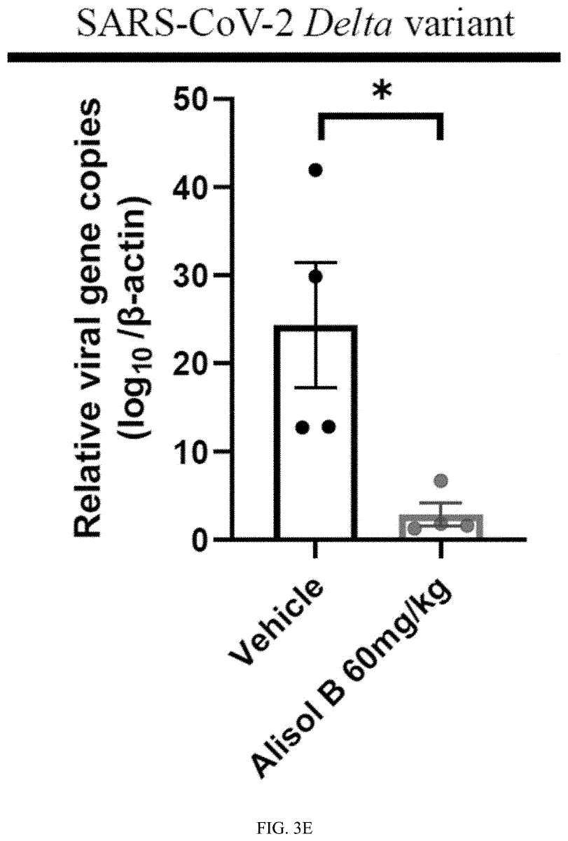

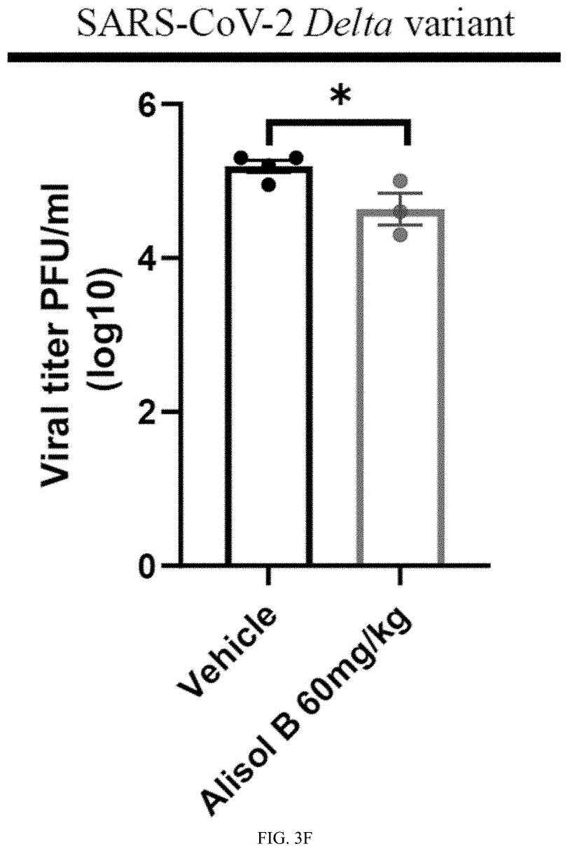

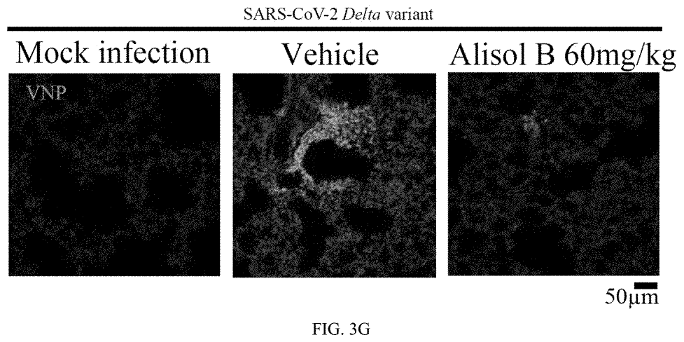

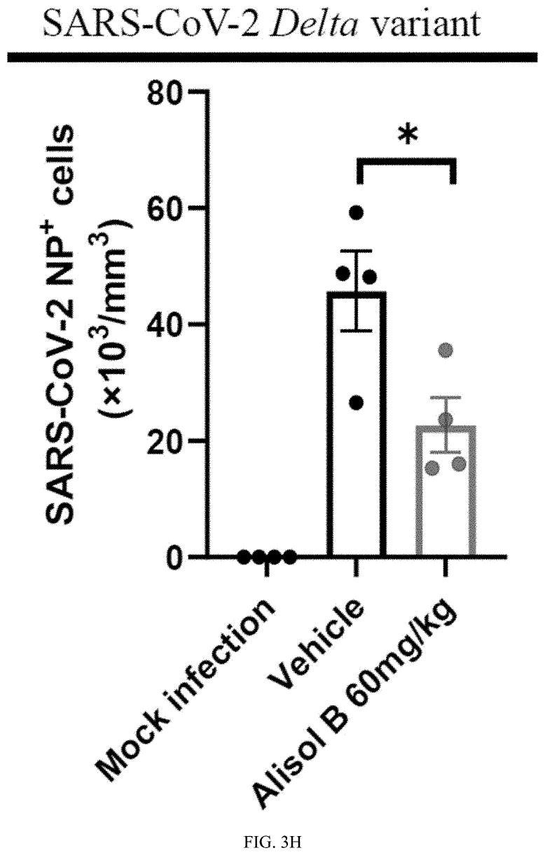

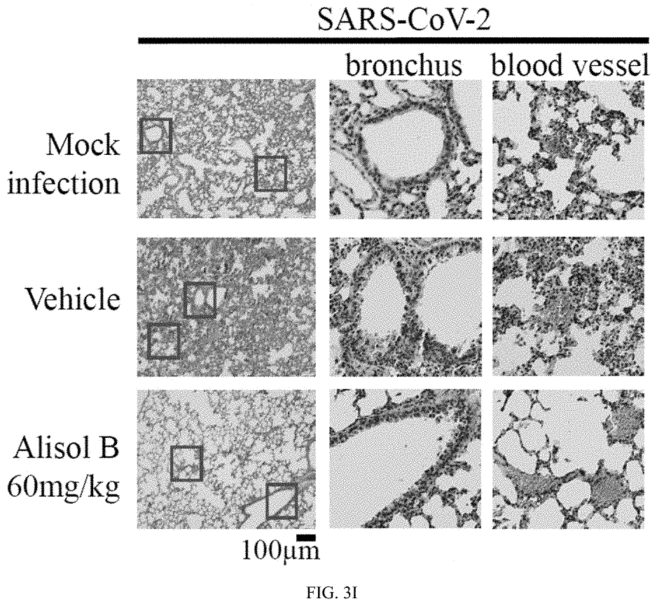

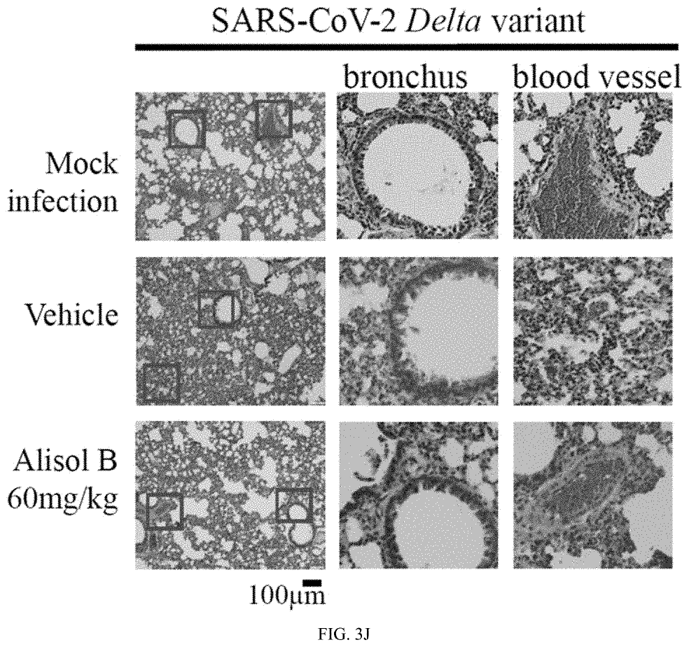

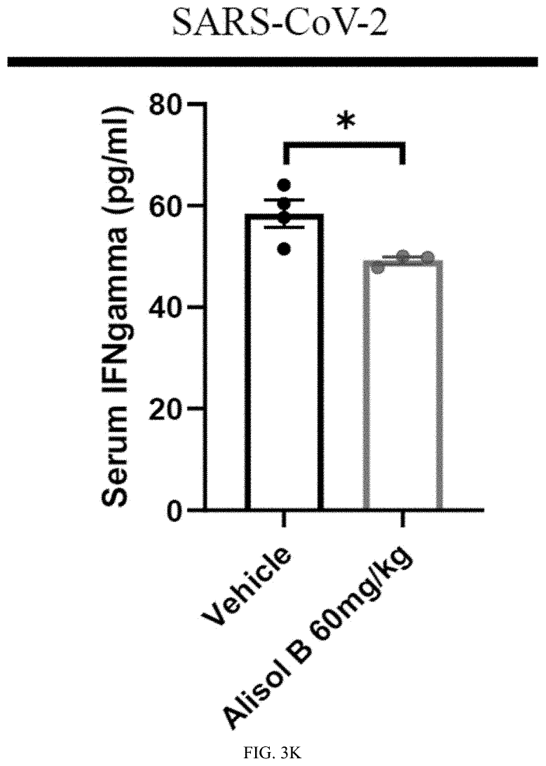

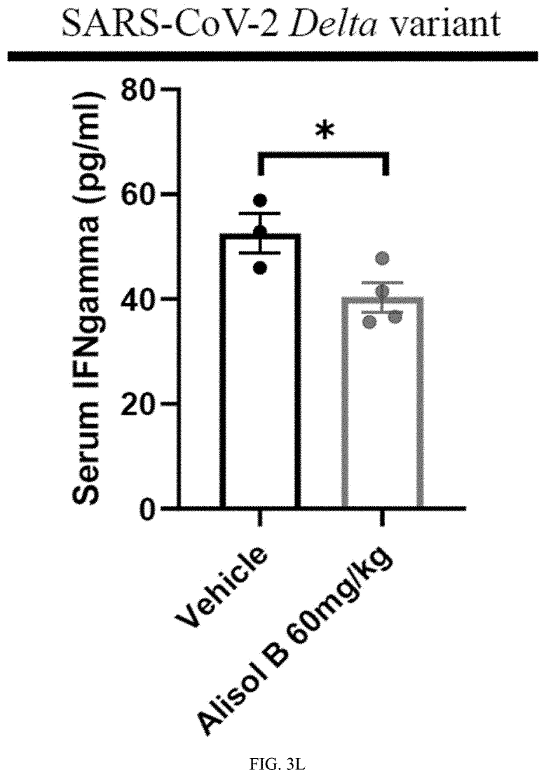

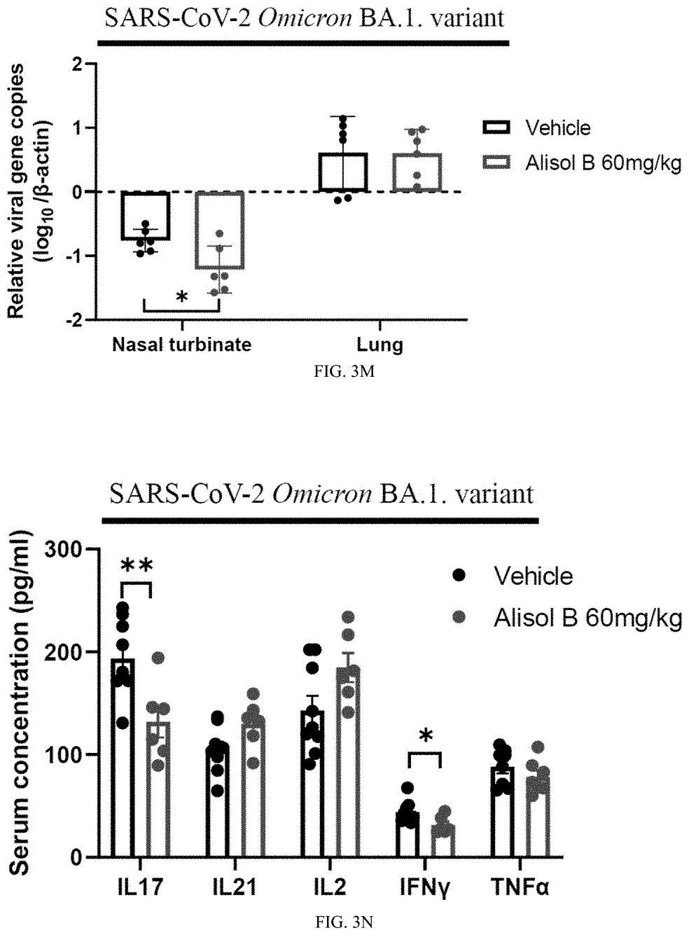

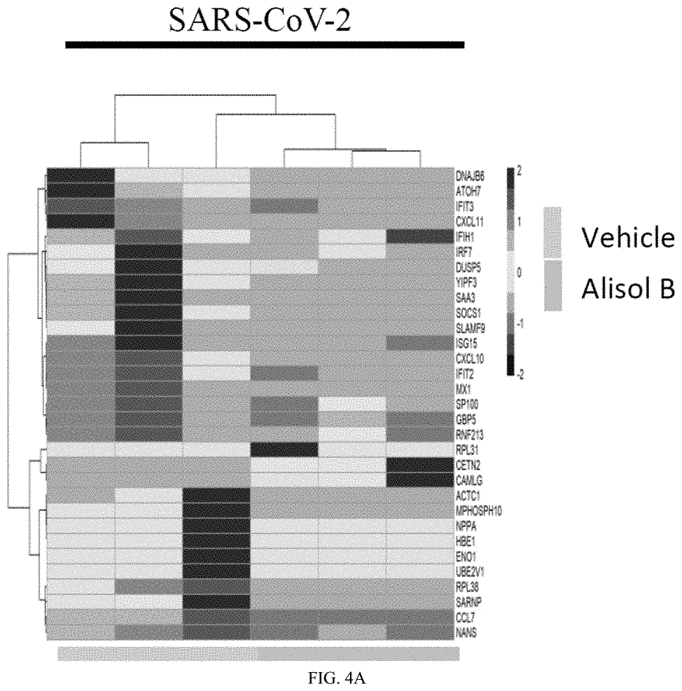

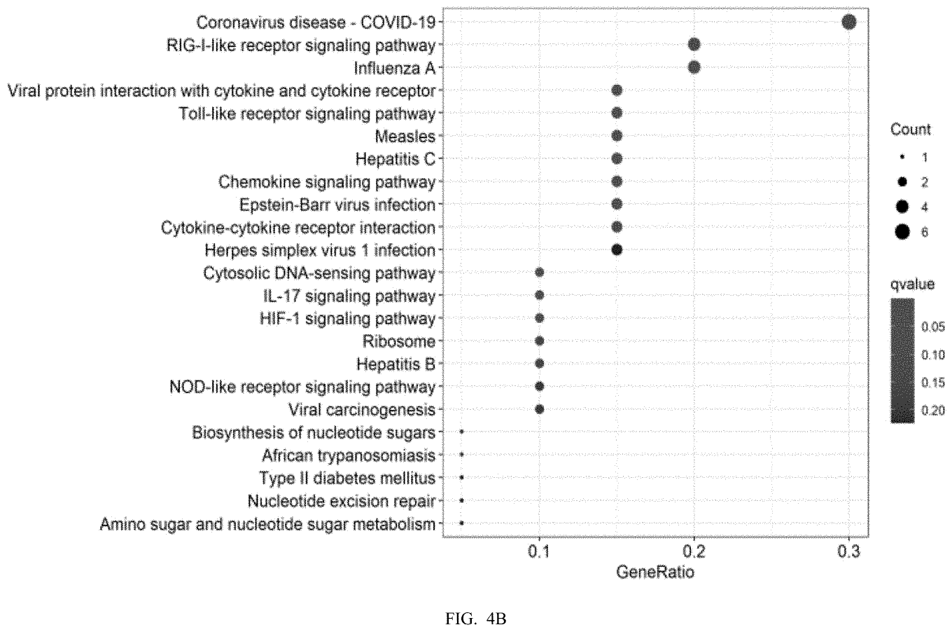

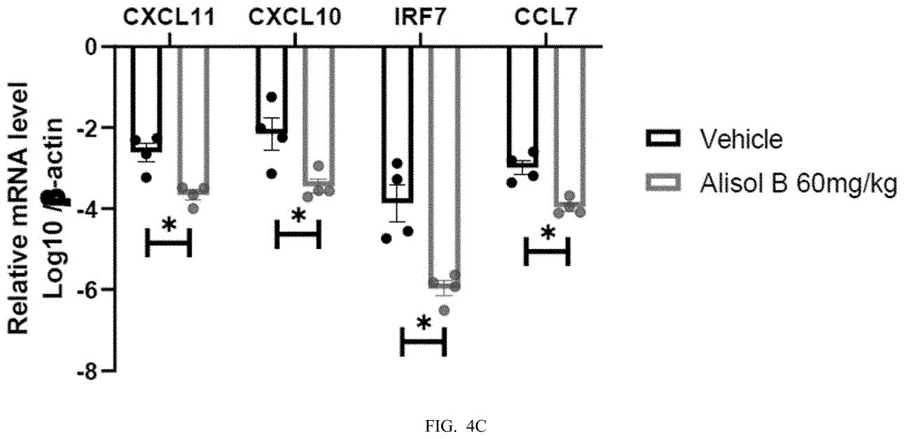

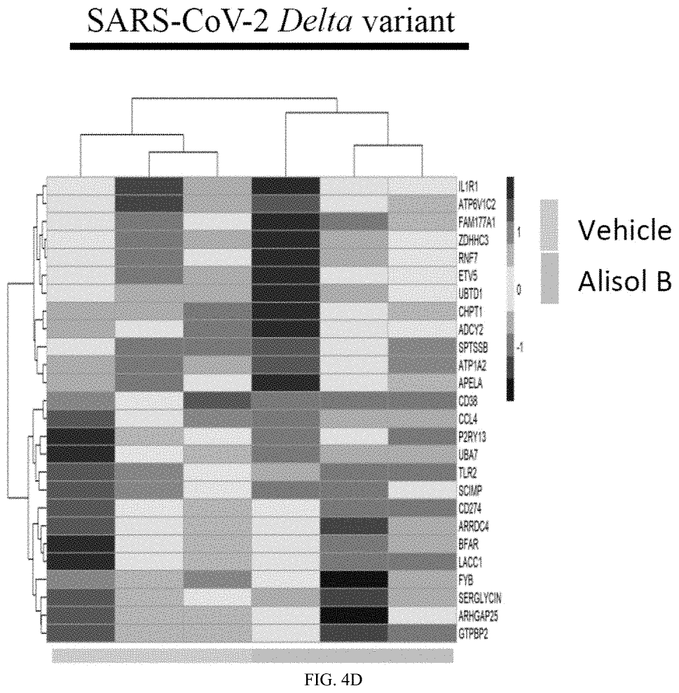

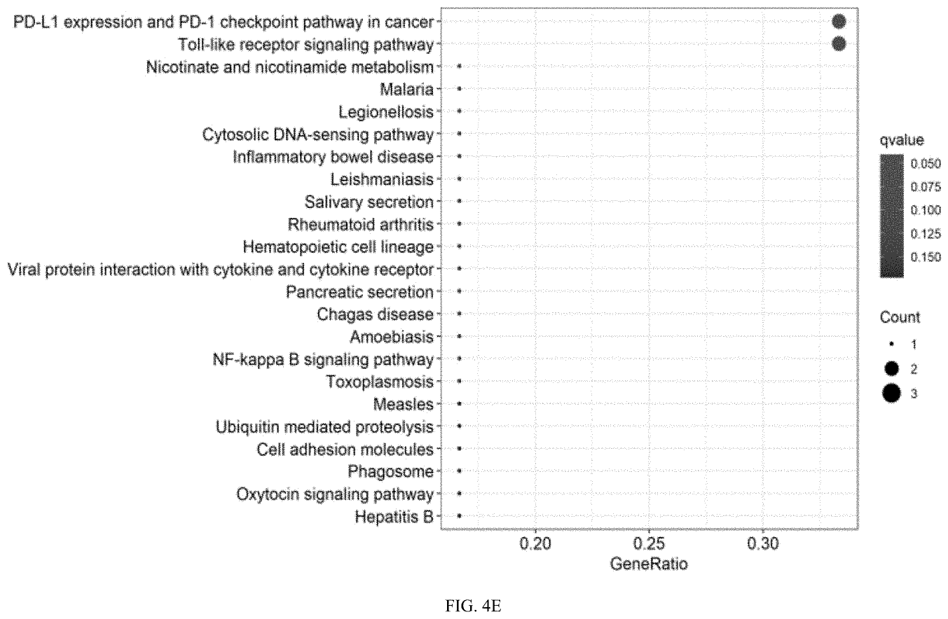

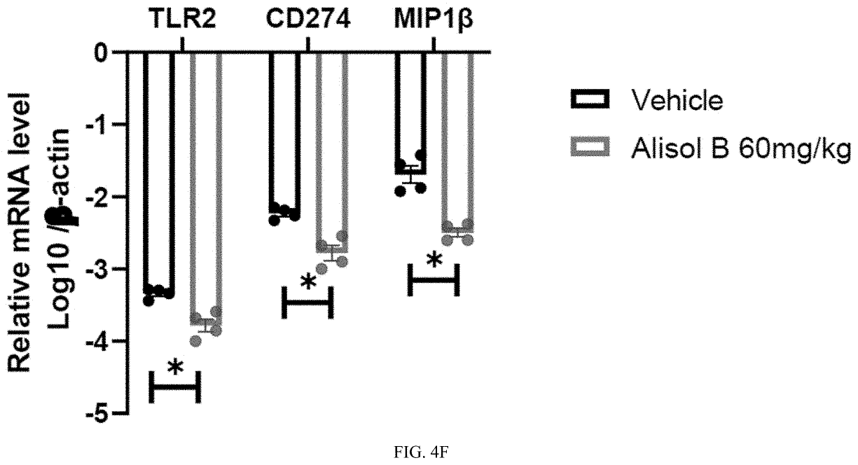

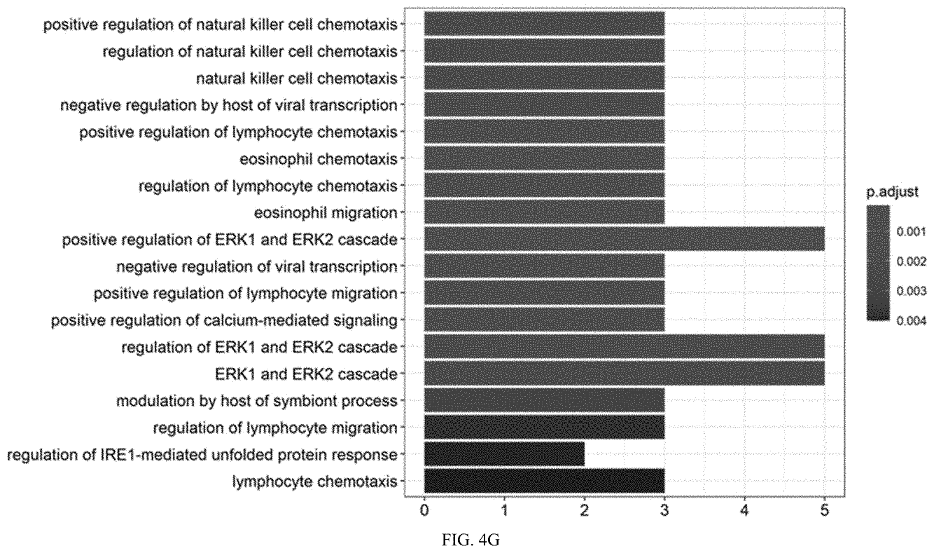

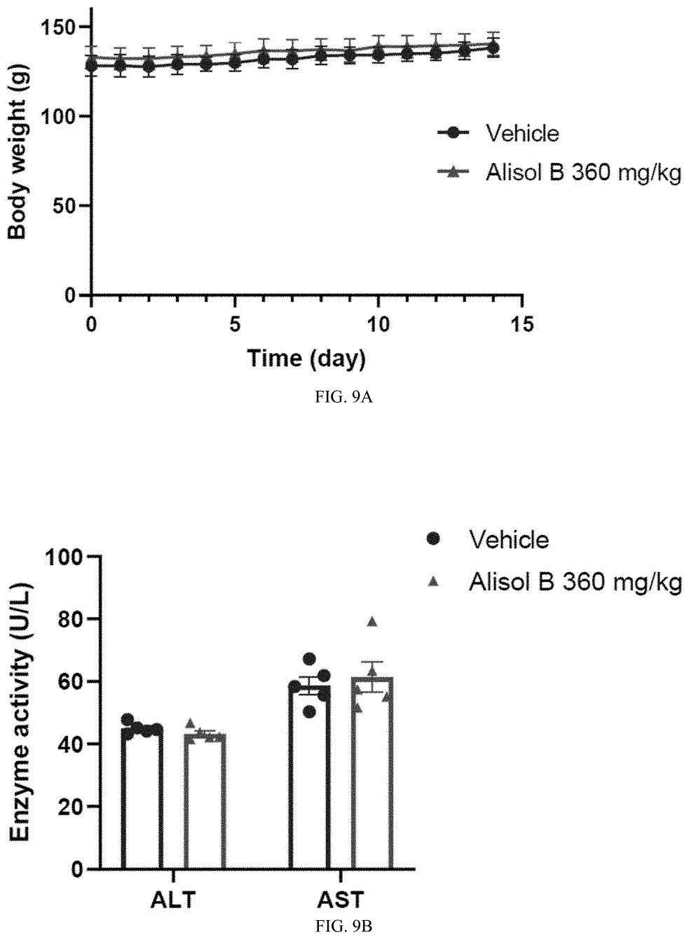

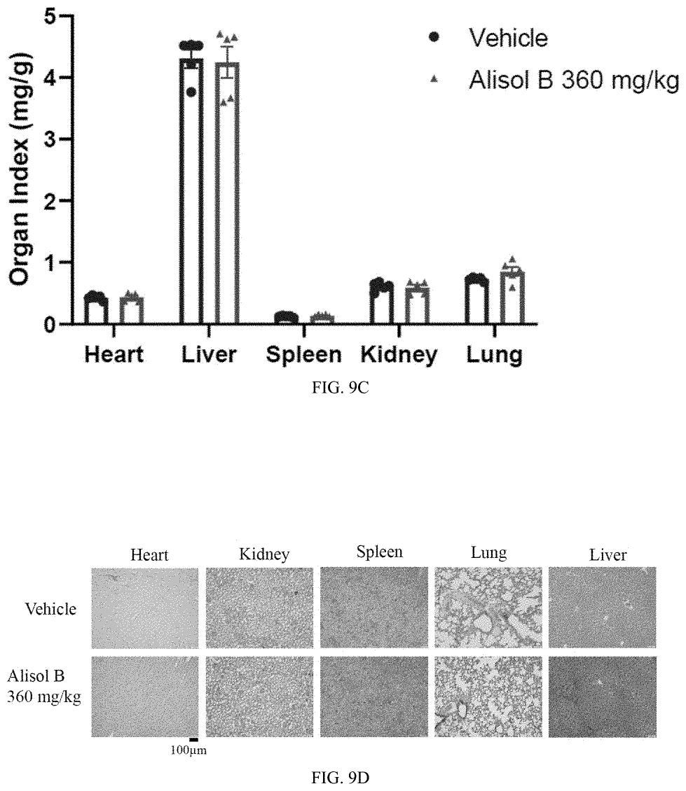

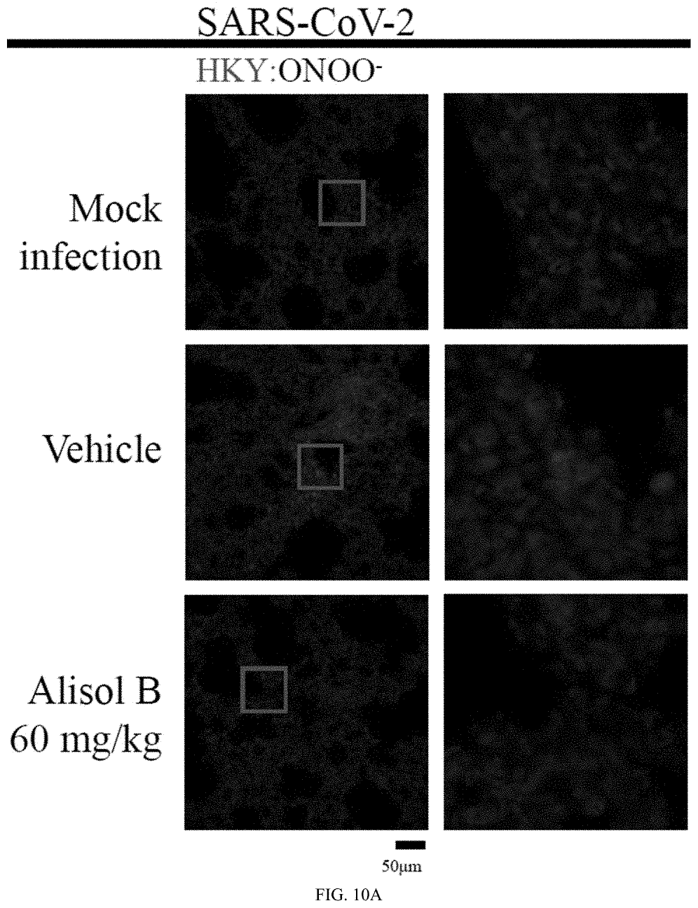

Here, we employed a golden Syrian hamster model[13] to test the antiviral efficacy of Alisol B. Alisol B (60 mg/kg) was intraperitoneally administrated into the hamsters. The dosage of Alisol B for hamster treatment was 60 mg/kg/day based on the toxicity studies. The dosage of toxicity study was choice at 420 mg/kg for hamster with the maximum concentration in solvent system. The solvent system was ethanol, PEG400 and saline (10:3:2) at 60 mg/mL due to the poor water solubility of Alisol B that used as vehicle. The results of body weight changes showed that Alisol B 360 mg/kg has no acute toxicity for the hamster (FIGS. 9A-9D). The results revealed that viral loads in the hamster lung were reached to the maximum companied with significant histopathological changes after the hamsters were infected by SARS-CoV-2 and SARS-CoV-2 delta variant. Alisol B treatment significantly reduced viral copies of SARS-CoV-2 and SARS-CoV-2 delta variant and plaque forming units in the lung tissues (FIGS. 3A-3H). The RNA sequence results showed the similar results that the viral transcription was inhibited by Alisol B treatment (FIGS. 4A-4F). 34 genes were significantly regulated after Alisol B treatment. All the genes with differential expression were processed to the GO analysis. From FIG. 4G, the major biological processes regulated by Alisol B were “positive regulation of NK cells chemotaxis” and “negative regulation by host of viral transcription”. The main target of Alisol B might be the ERK1 and ERK2 associated pathway from the results of GO analysis of biological process. We also detected the virus titer in nasal wash solution. Thus, Alisol B treatment antagonizes SARS-CoV-2 and delta variant replication in the lung tissues and reduces virus shedding in feces.

### Example 3—23-O-Acetylalisol B has Anti-Inflammation and Antioxidant Effects Against SARS-CoV-2 and SARS-CoV-2 Delta Variants Induced Lung Damages in Hamster Covid-19 Model

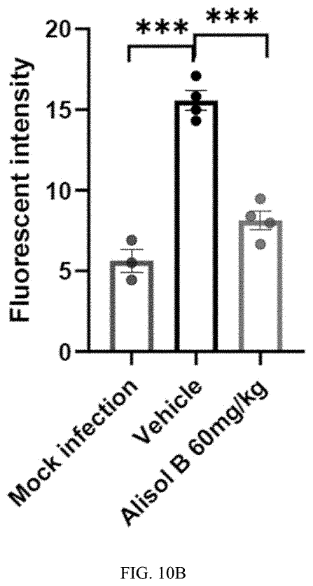

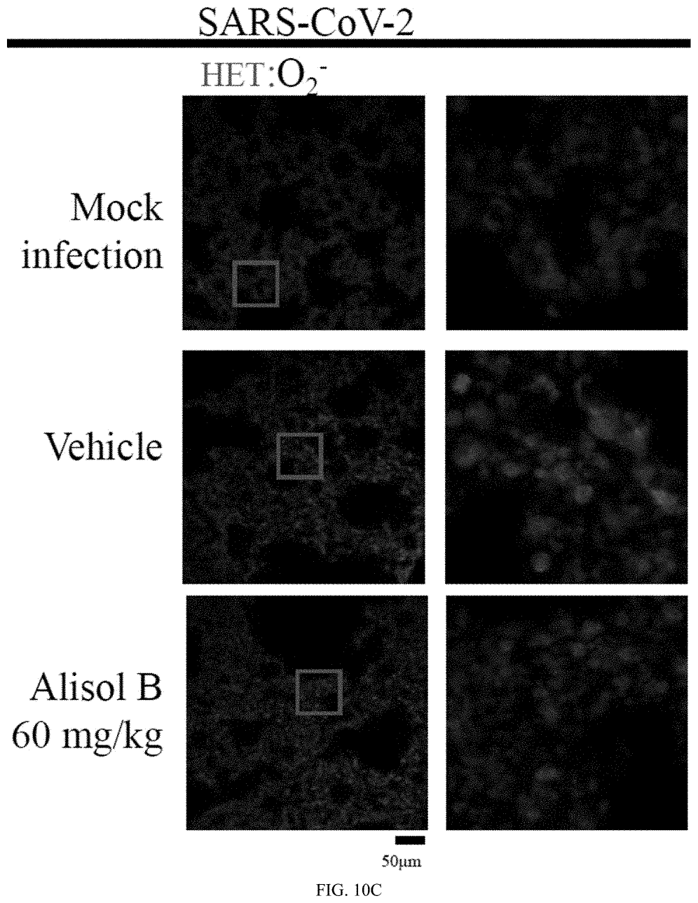

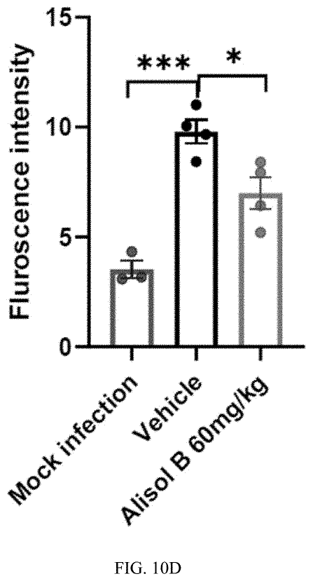

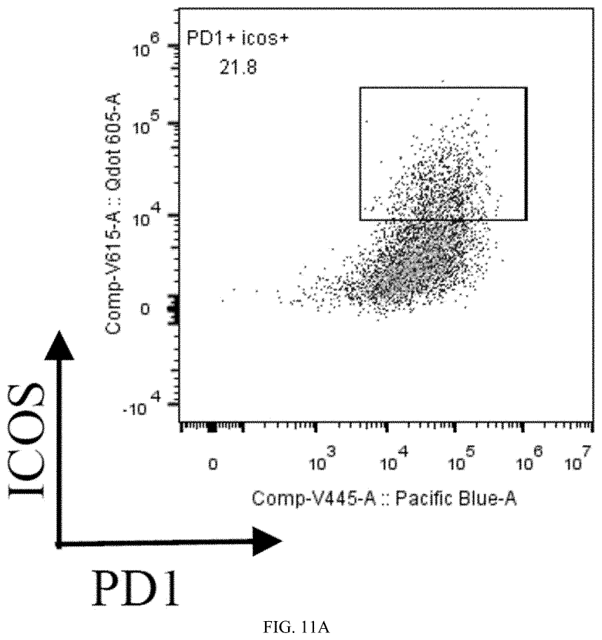

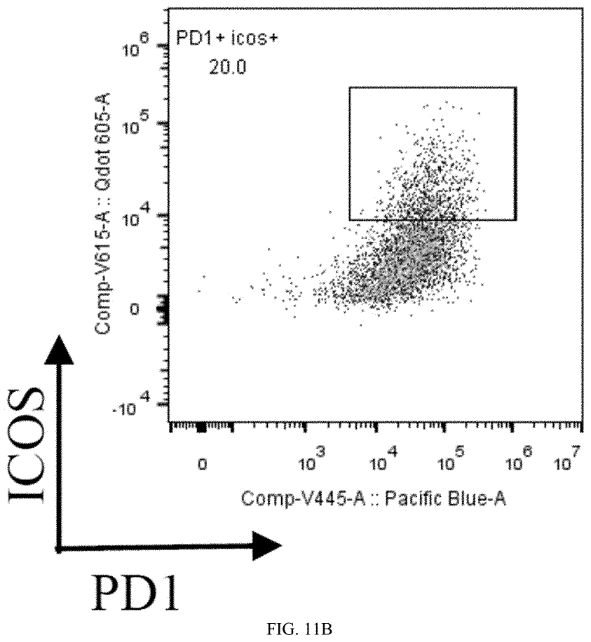

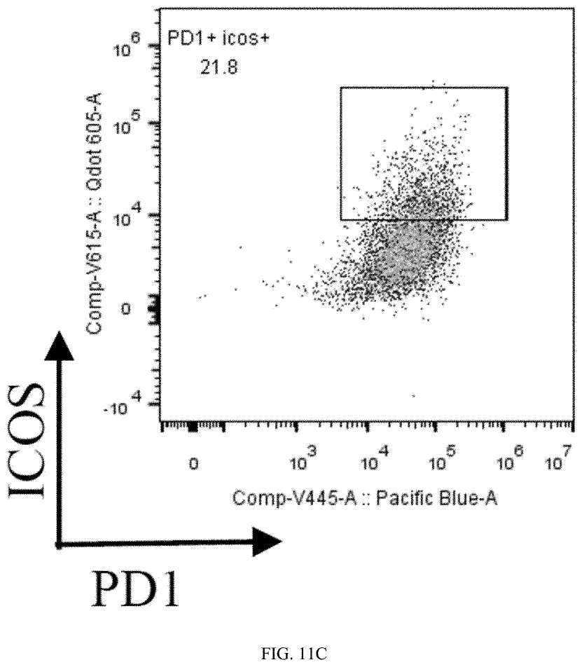

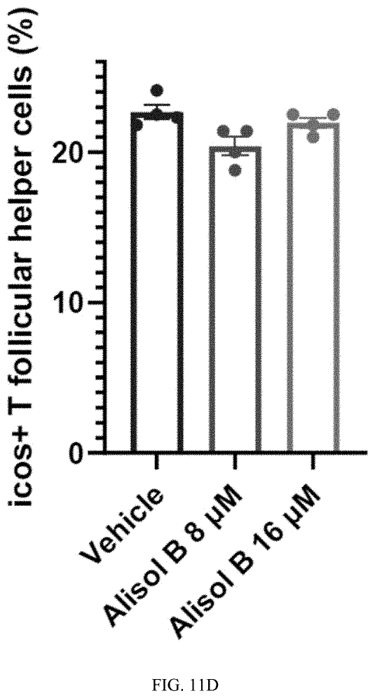

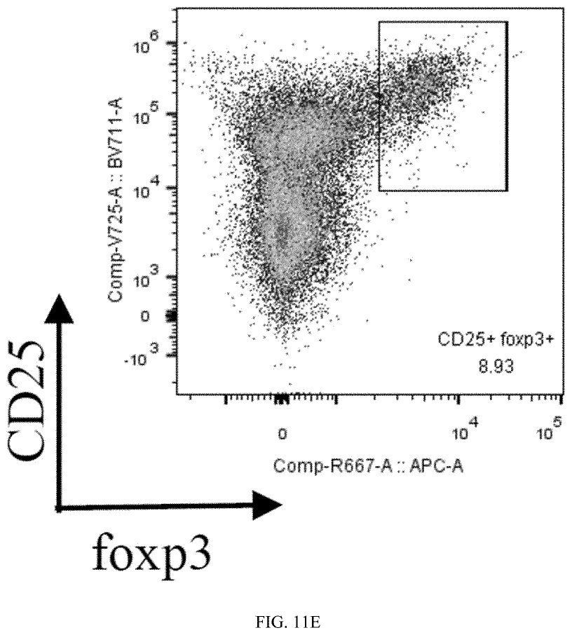

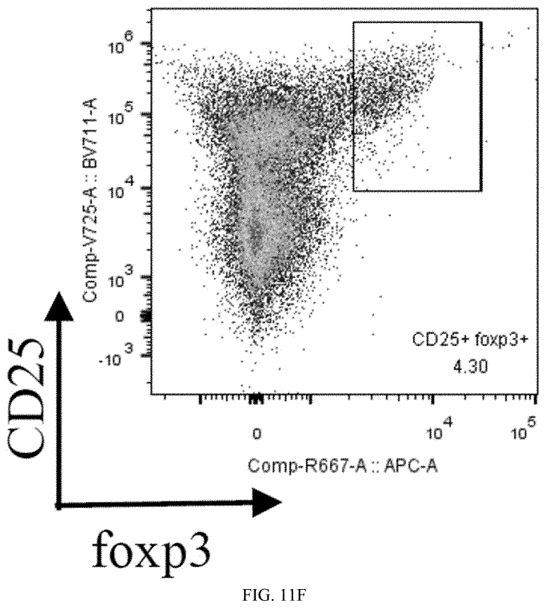

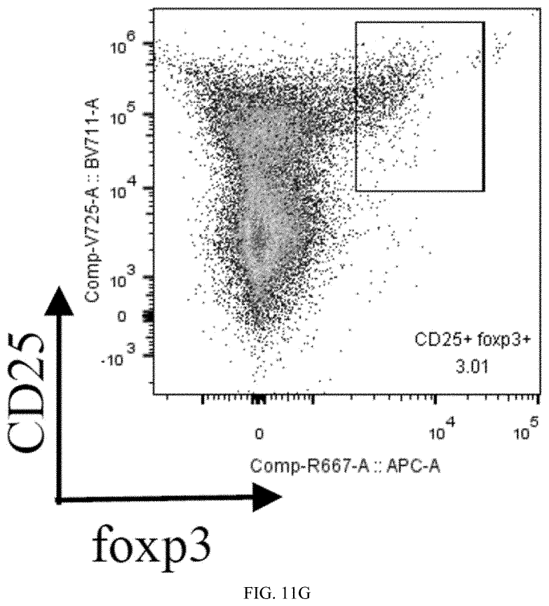

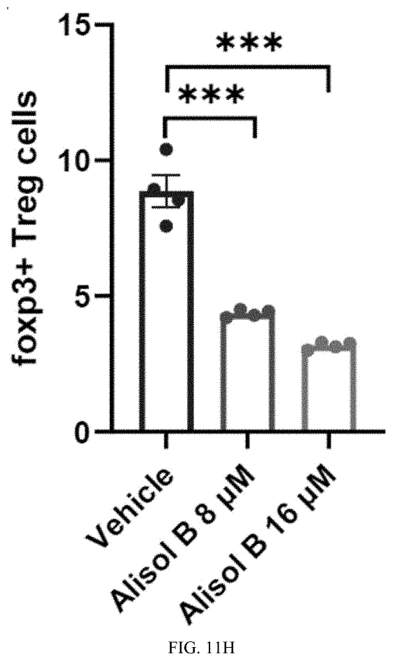

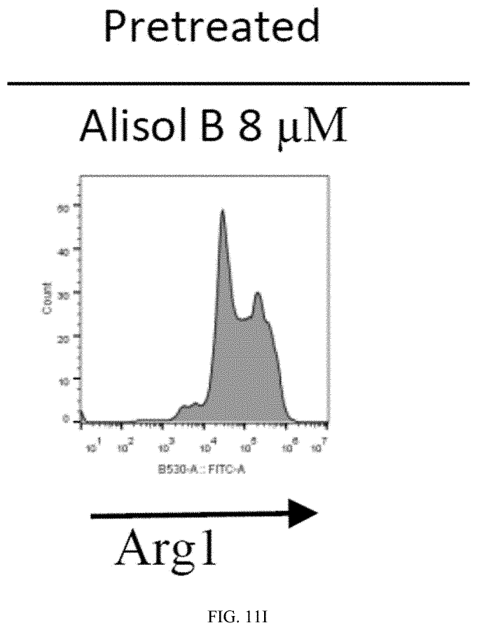

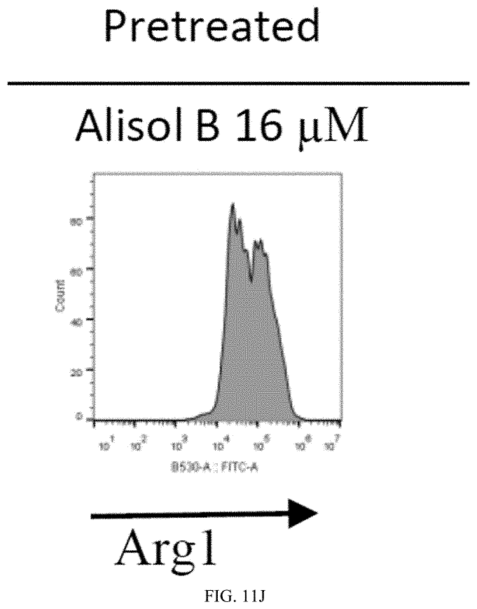

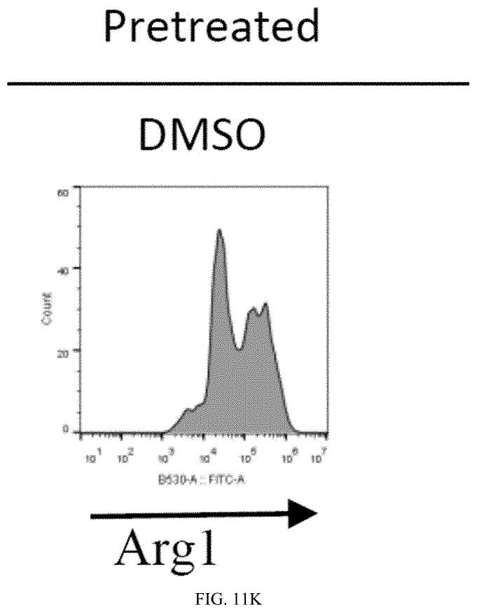

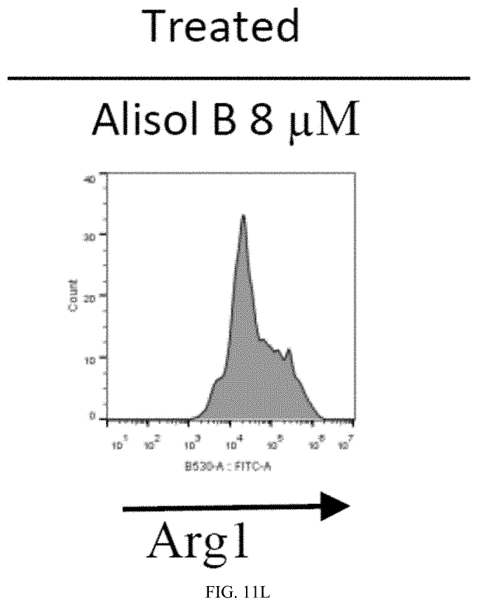

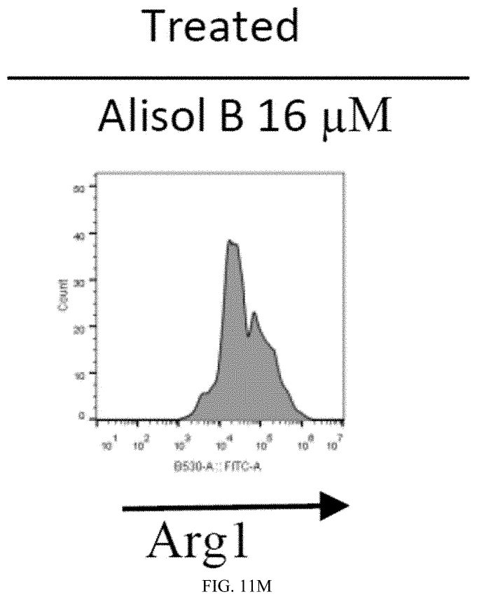

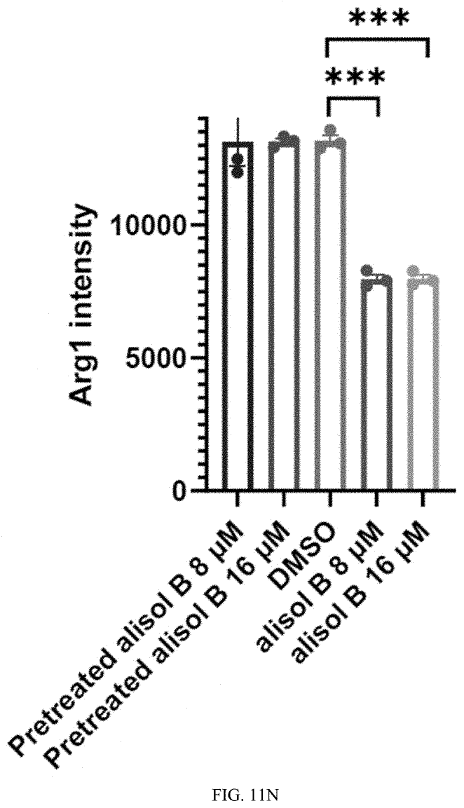

We then tested hypothesis that Alisol B could have anti-inflammation and antioxidant effects, and attenuate respiratory failure syndromes against SARS-CoV-2 and SARS-CoV-2 Delta variants induced lung damages. Hematoxylin and eosin staining was used for histological examination of the lung tissues. When compared with vehicle treatment group, Alisol B treatment group had significantly reduced pathological changes and showed lower expression level of inflammation cytokines in the lung tissues with less consolidation and cell infiltrations in blood vessel and peribronchiolar area (FIGS. 3I-3L, 4C, 4F). To address the effects of Alisol B on T cells and microphage activation, we tested the serum interferon gamma (IFN-γ) level that is correlated to the pro-inflammatory innate immunity in COVID-19 [15]. Compared with vehicle control group, Alisol B treatment group had significantly lower level of serum IFN-γ in the hamsters with the infections of SARS-CoV-2 and SARS-CoV-2 Delta variants (FIGS. 3K-3L). Meanwhile, with the treatment of Alisol B, the expression levels of IL6 and IP10 (CXCL10) in lung were significantly decreased in the hamsters (FIGS. 4C and 4F). We also evaluated the antioxidant effects of Alisol B on the production of peroxynitrite and superoxide in the lung tissues. Alisol B treatment significantly reduced the levels of endogenous peroxynitrite and superoxide in lung tissues (FIGS. 10A-10D), subsequently inhibiting oxidative stress induced inflammation. Taken together, Alisol B has anti-inflammation, antioxidant and immunodulation effects, and protects against SARS-CoV-2 and SARS-CoV-2 delta variants induced lung damages.

### Example 4—-23-O-Acetylalisol B has Immunomodulation Effects Against SARS-CoV-2 and SARS-CoV-2 Delta Variants Induced Lung Damages in Hamster Covid-19 Model In Vivo

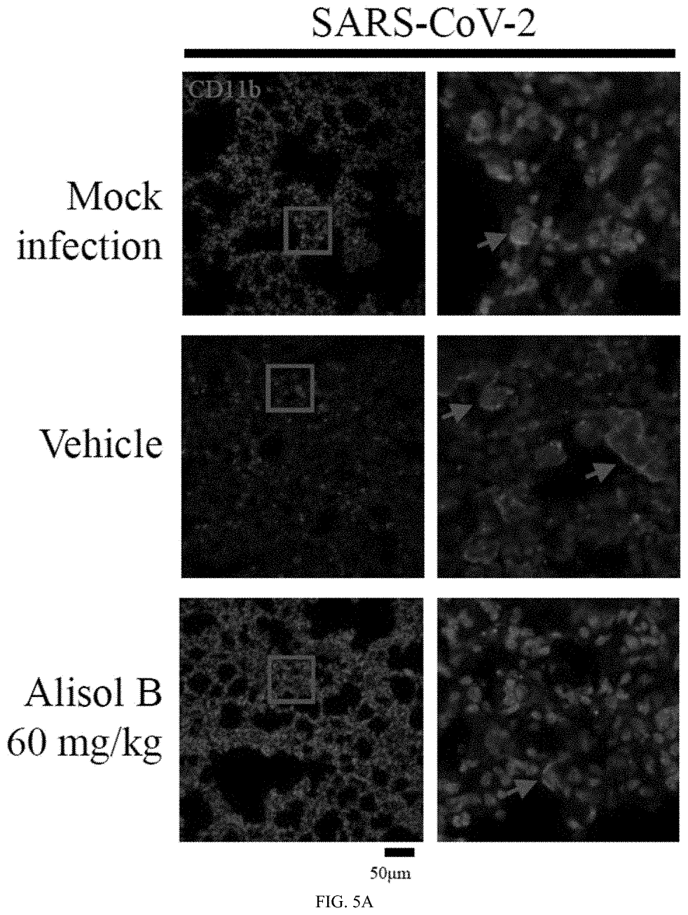

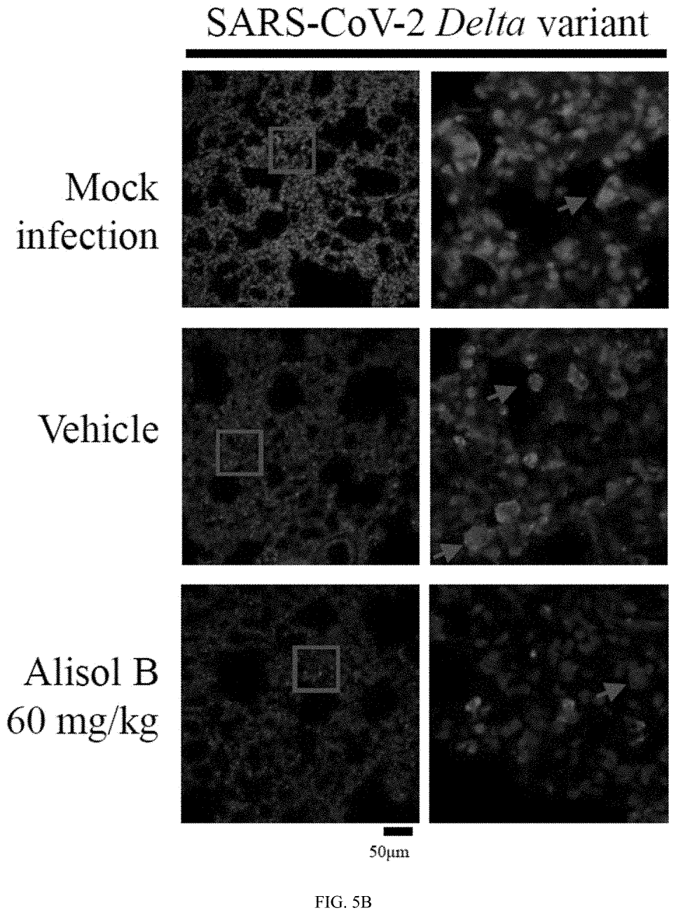

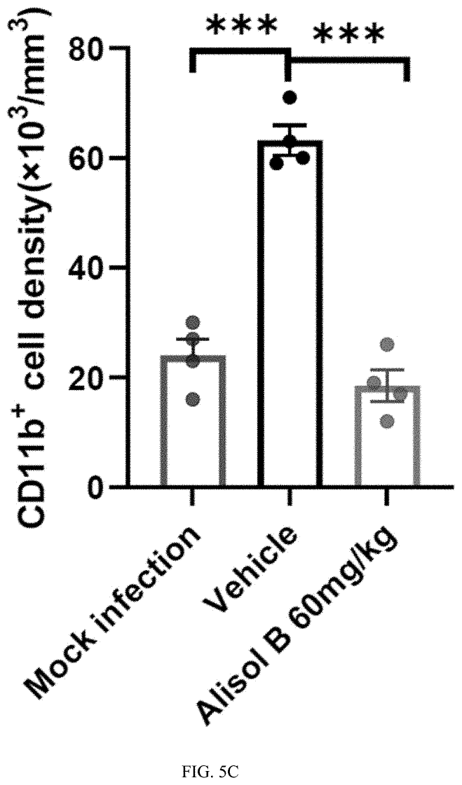

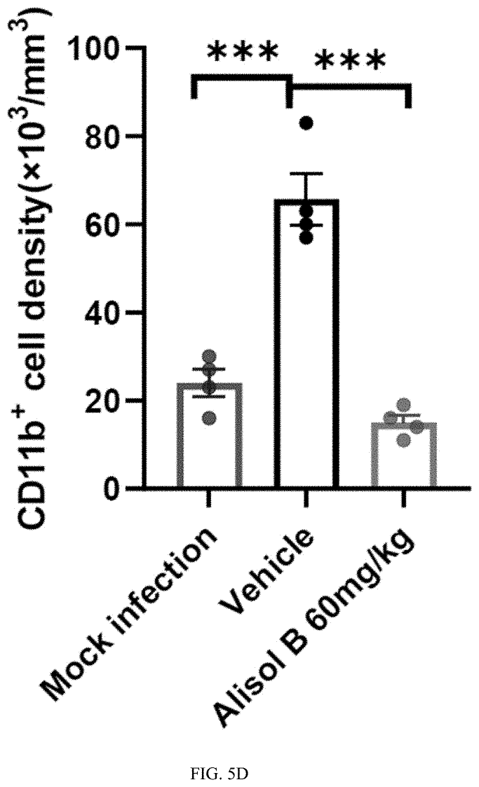

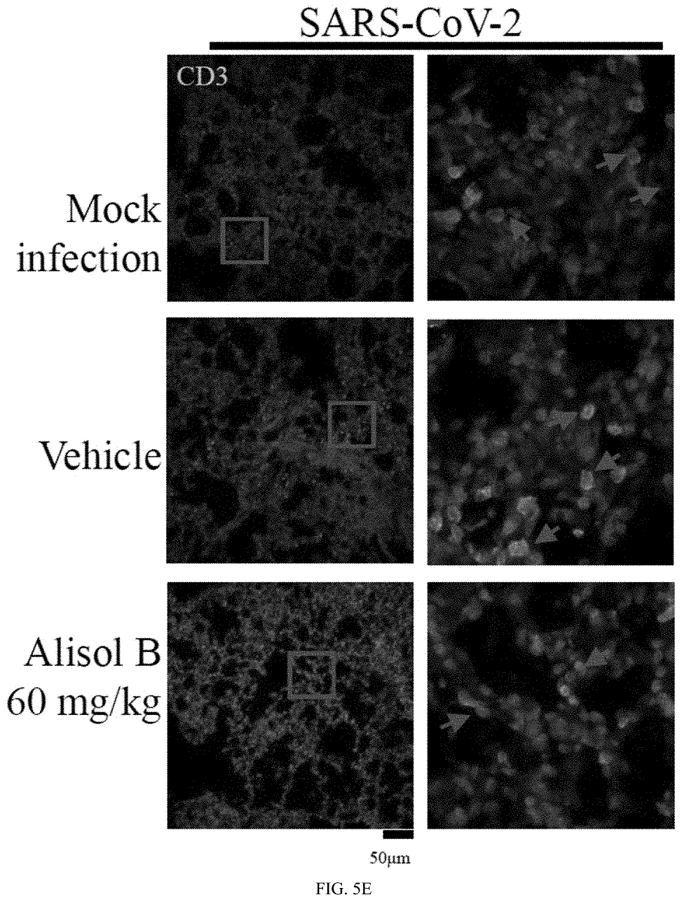

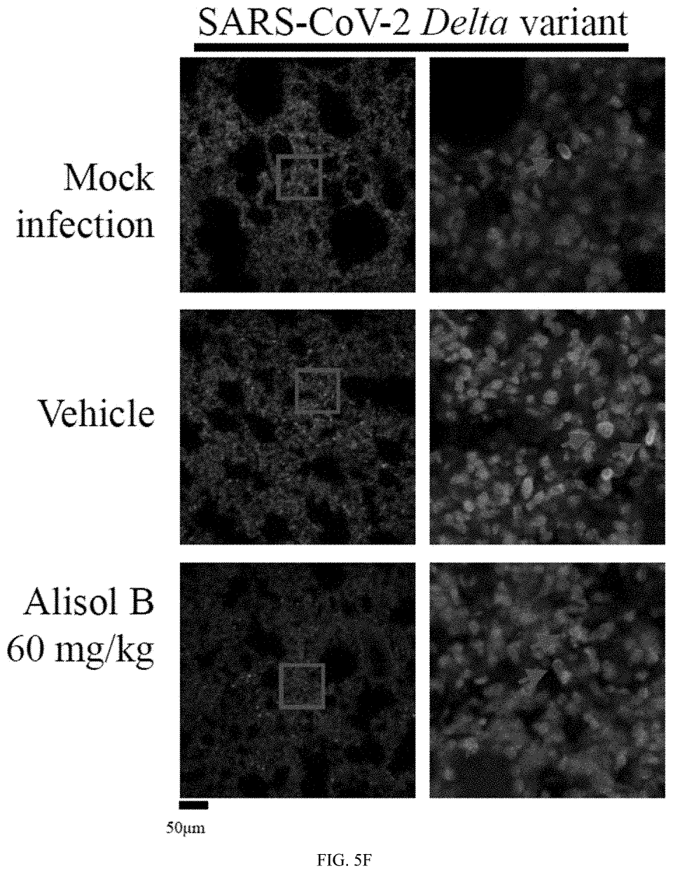

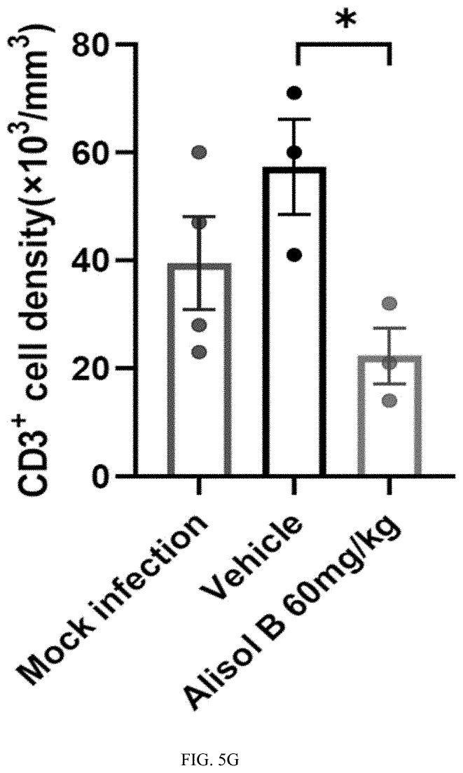

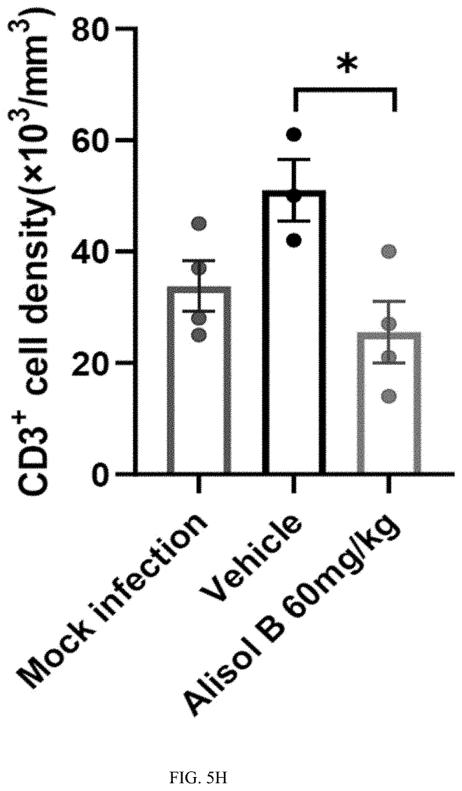

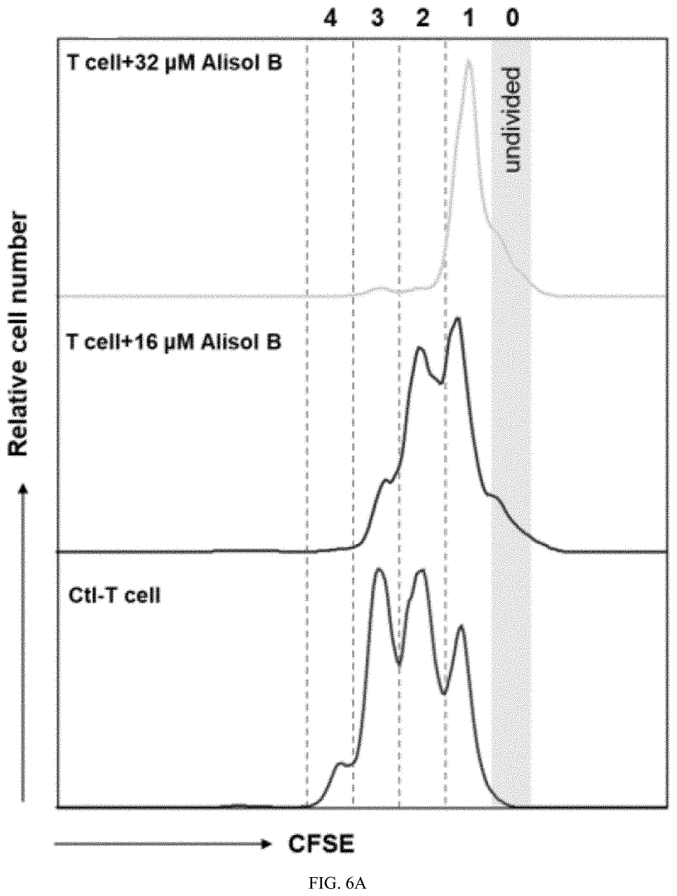

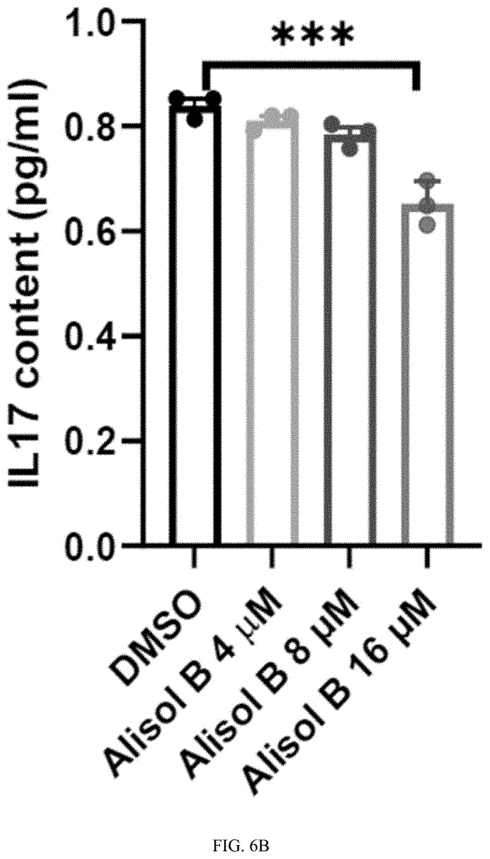

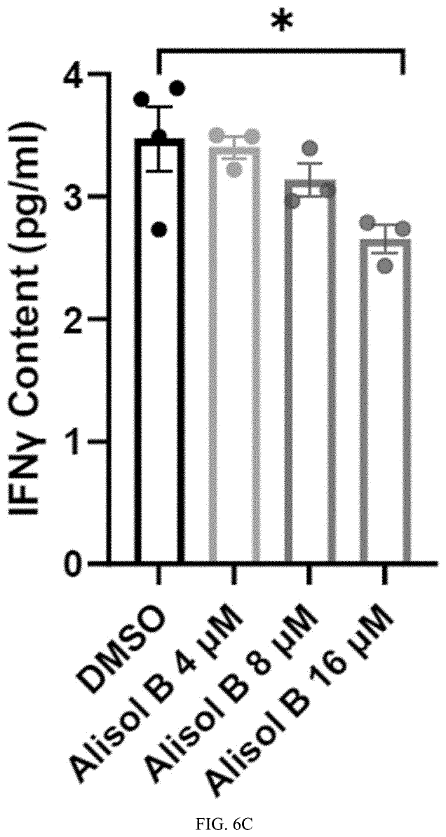

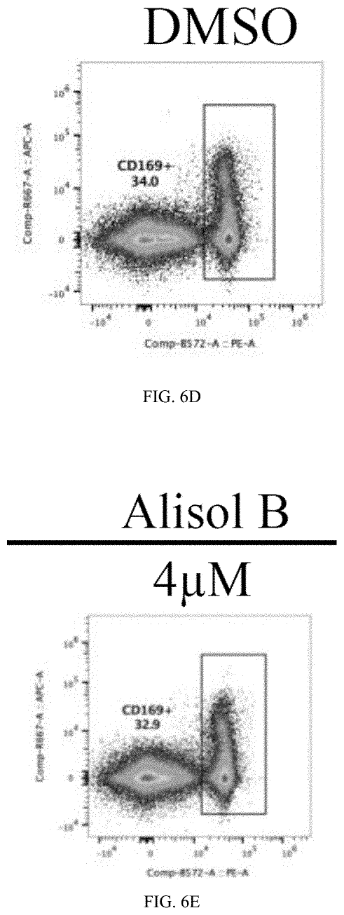

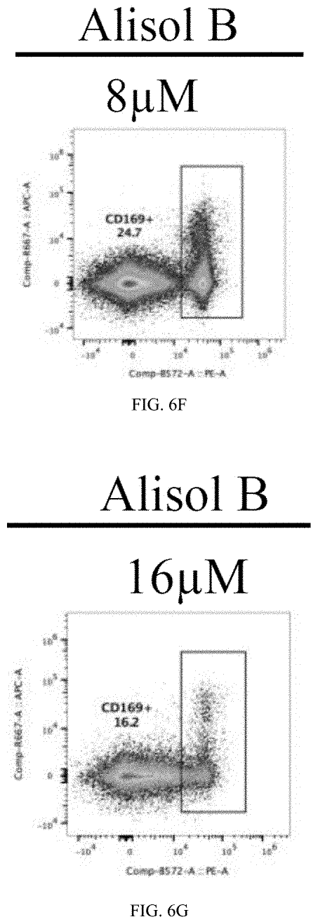

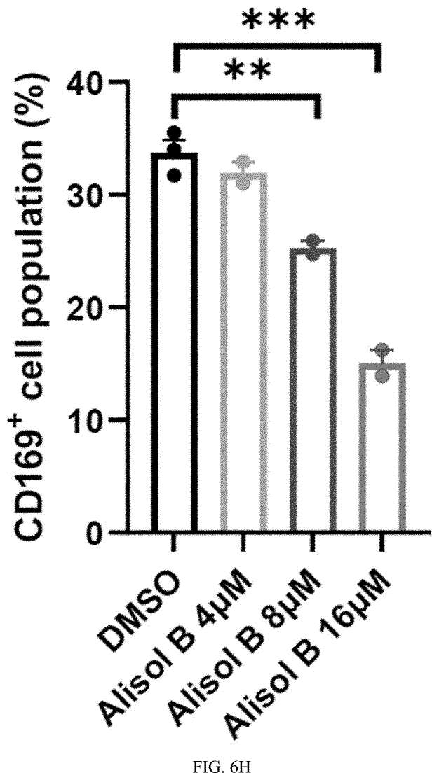

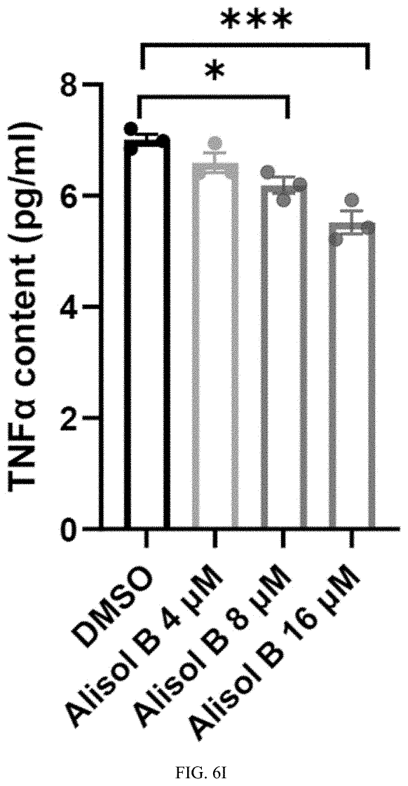

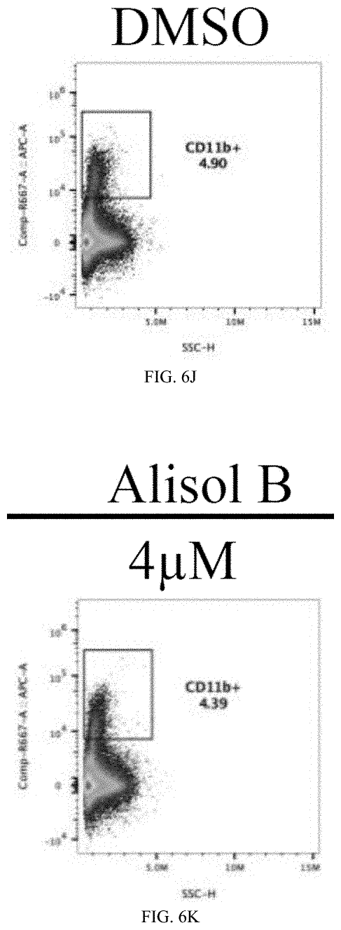

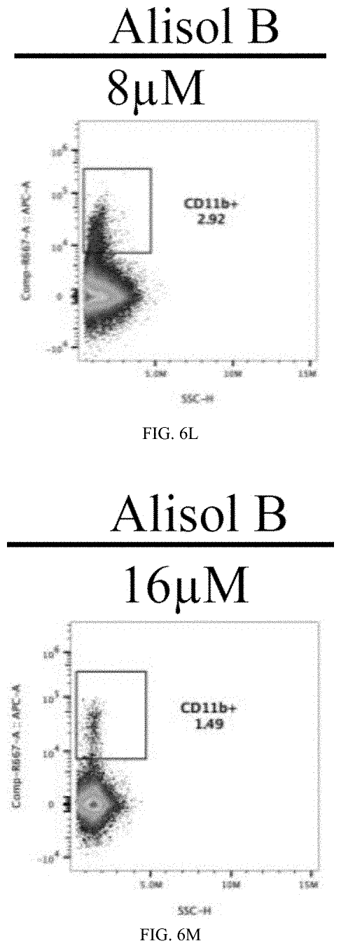

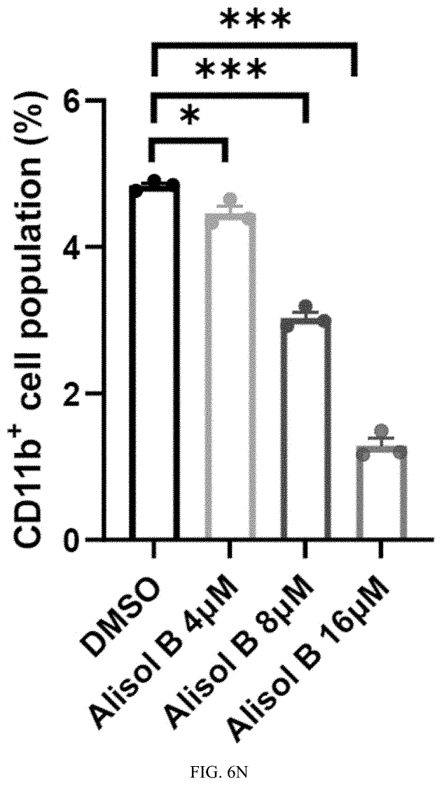

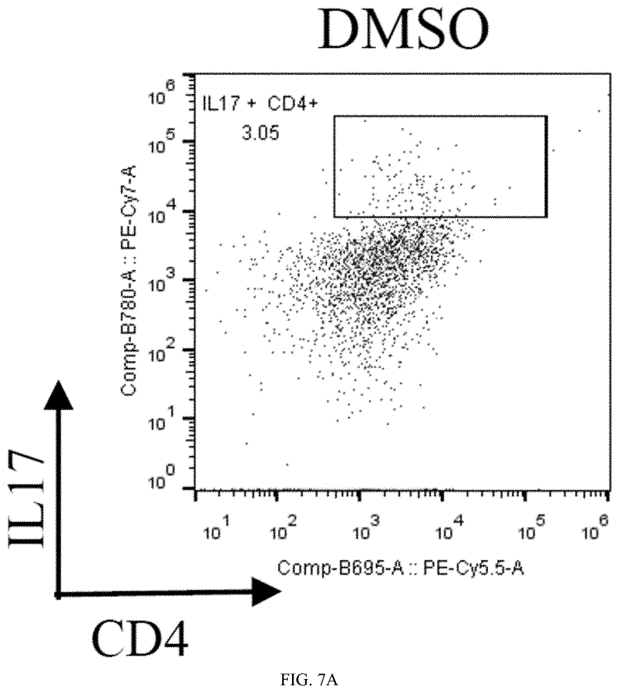

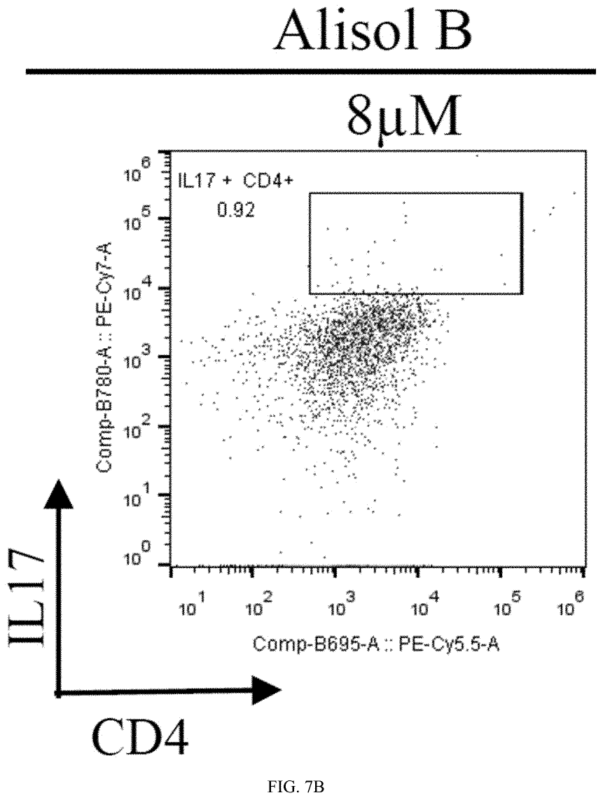

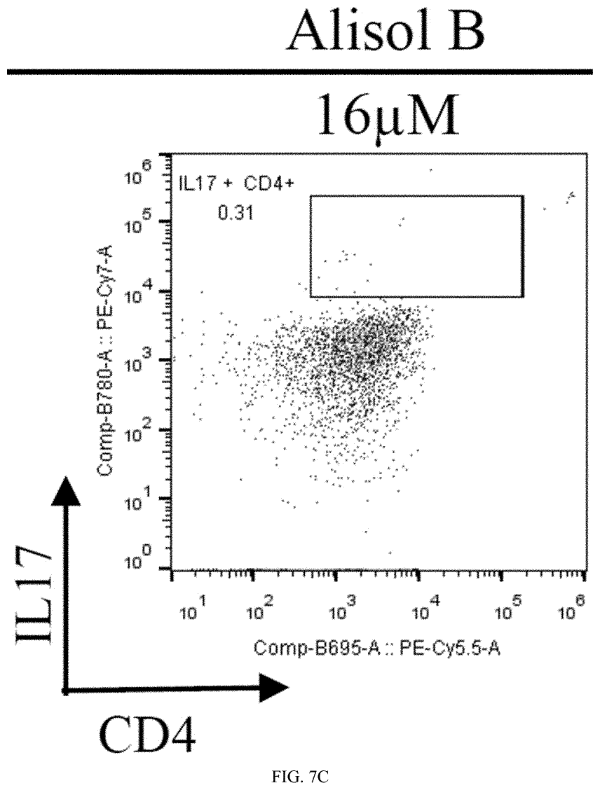

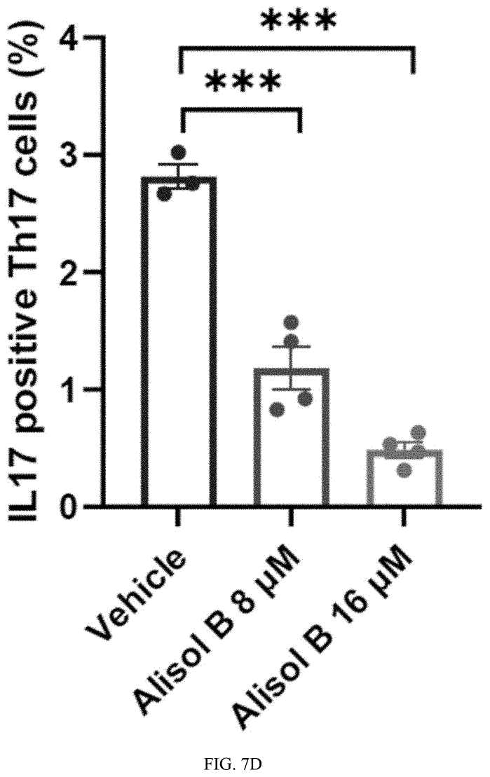

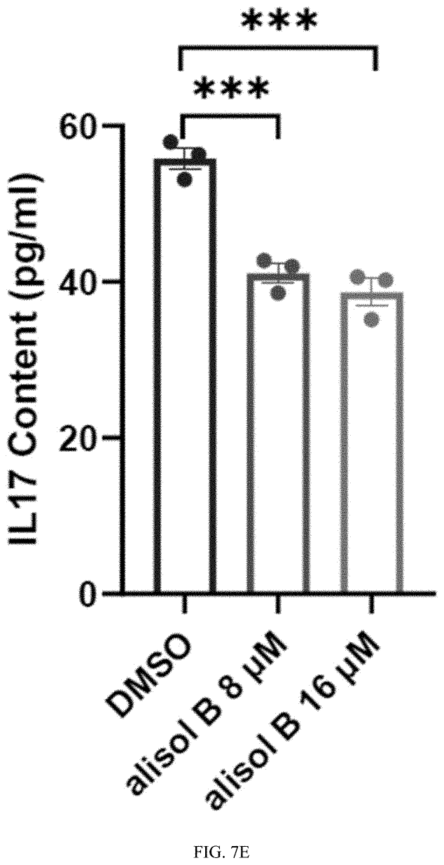

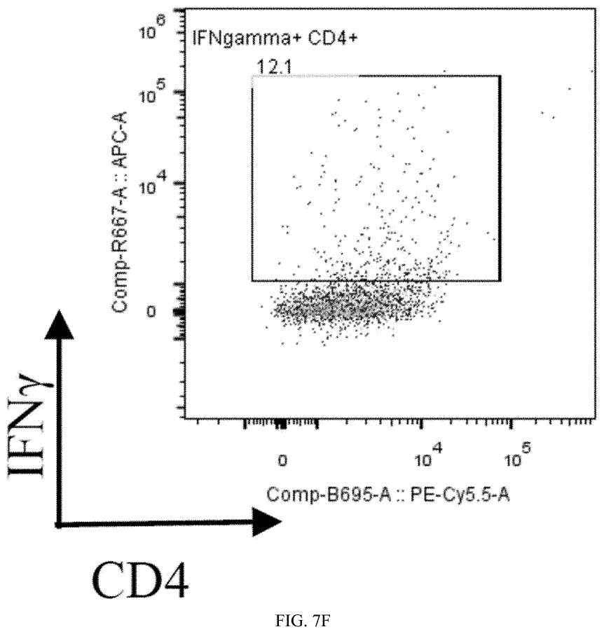

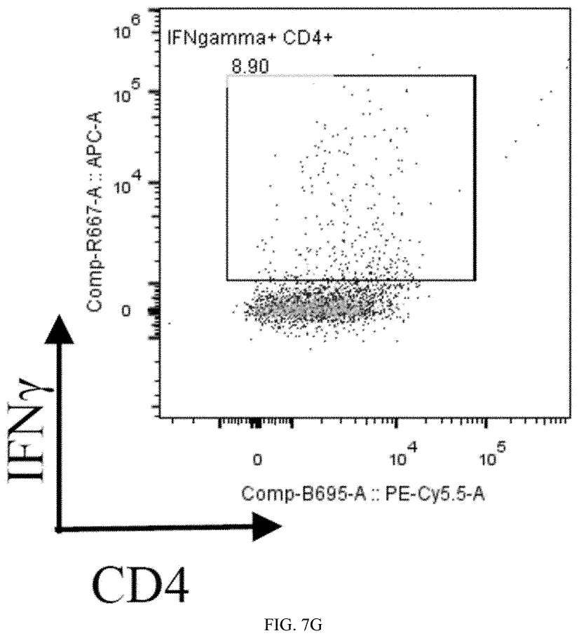

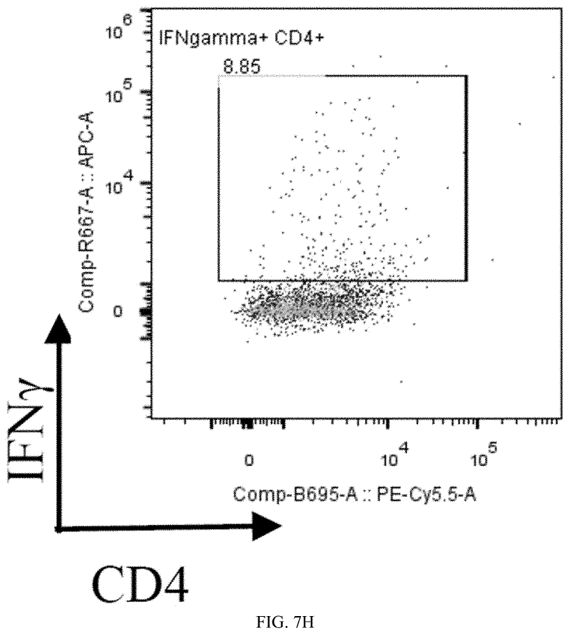

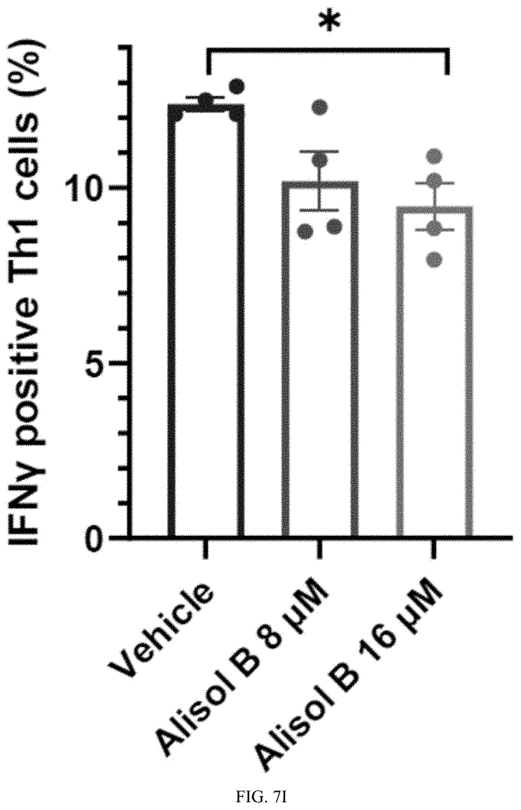

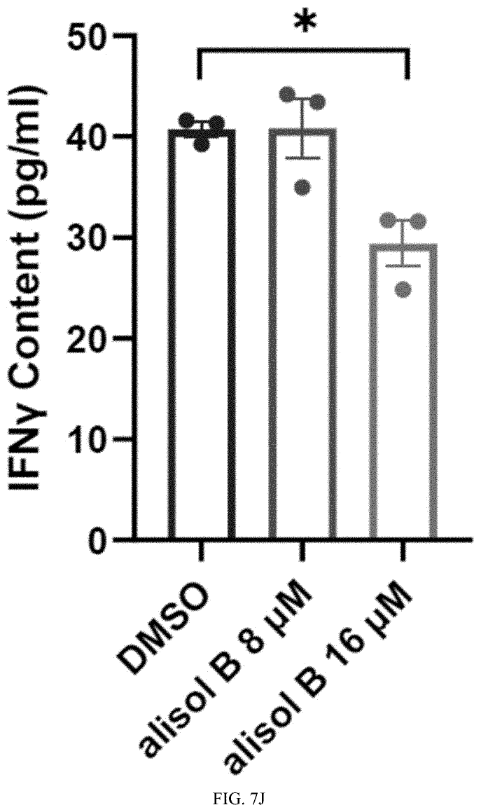

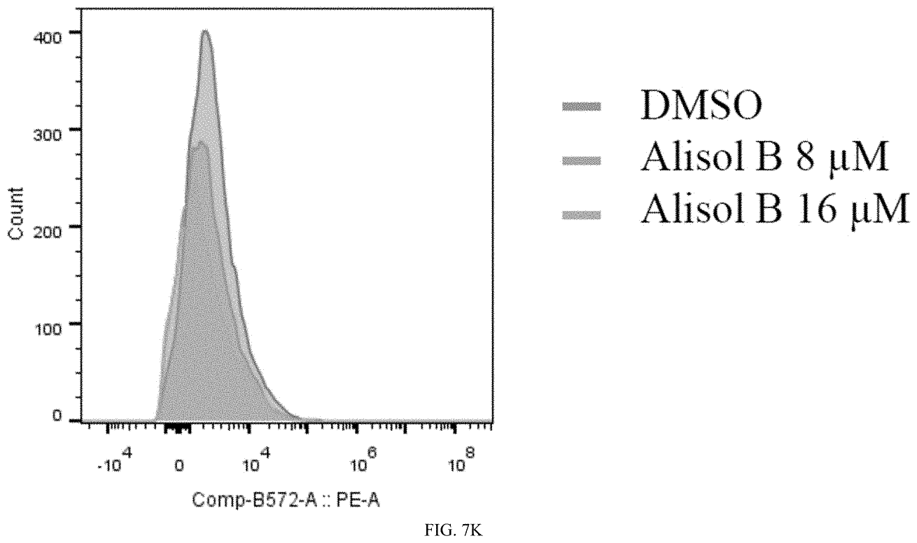

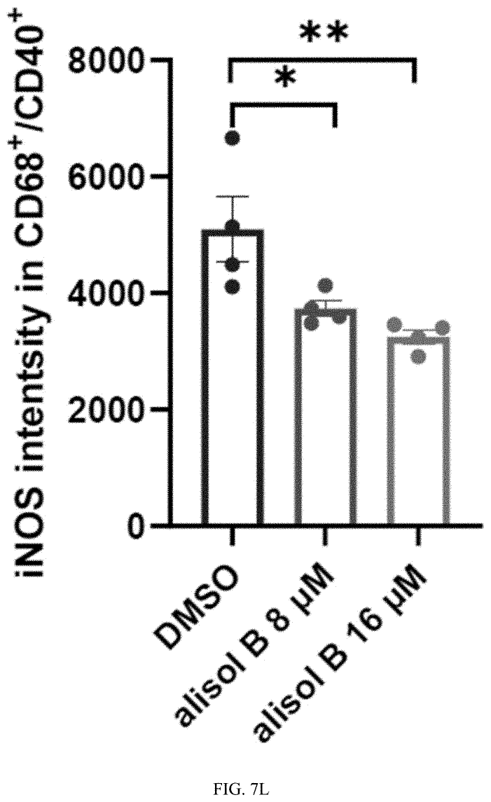

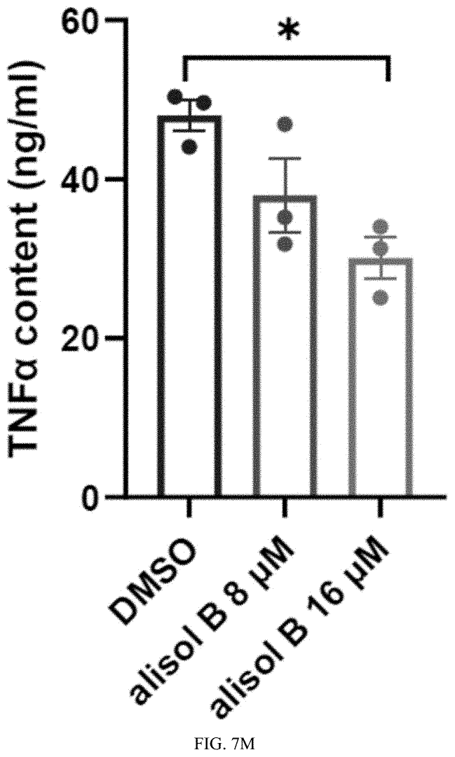

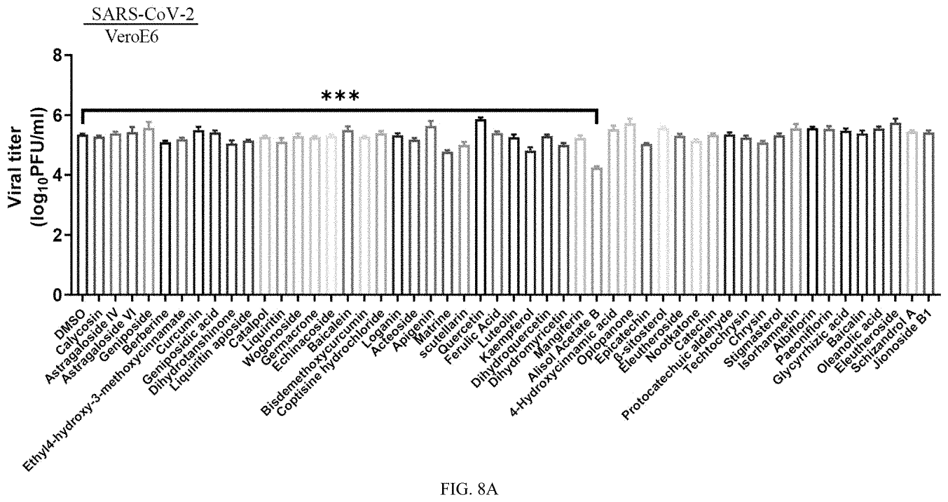

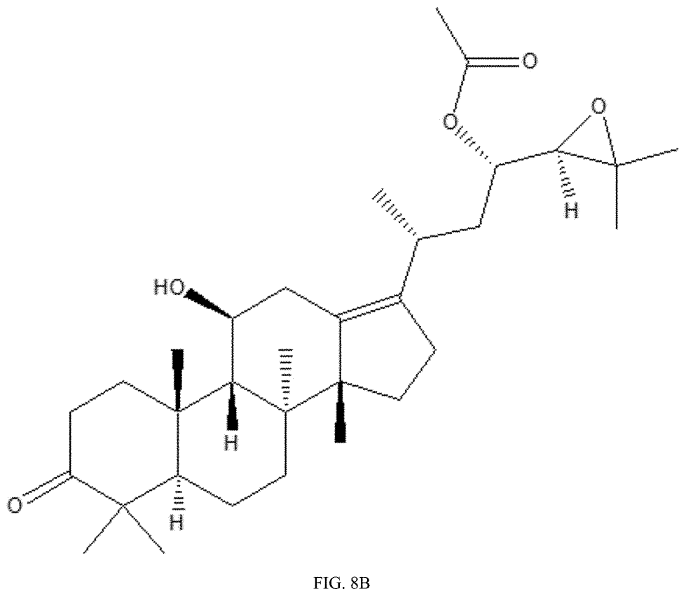

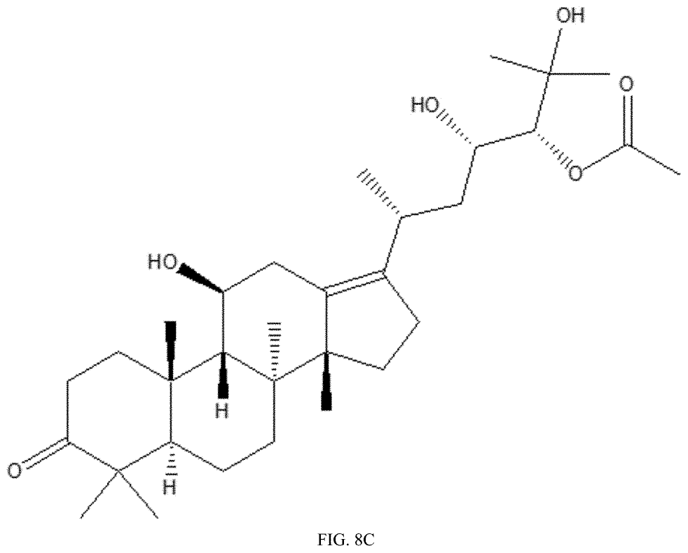

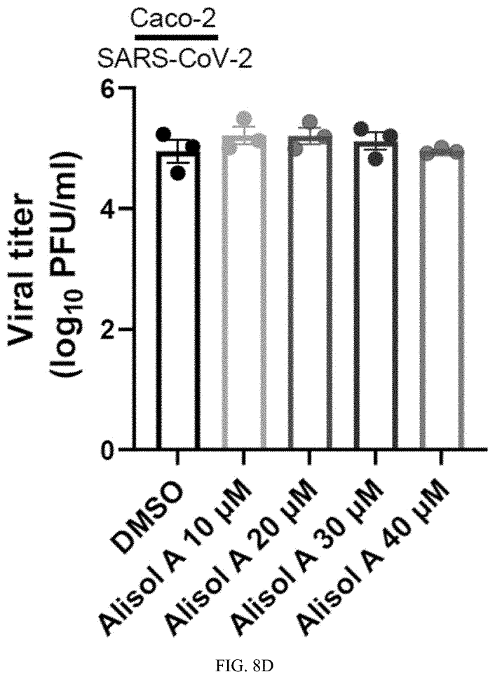

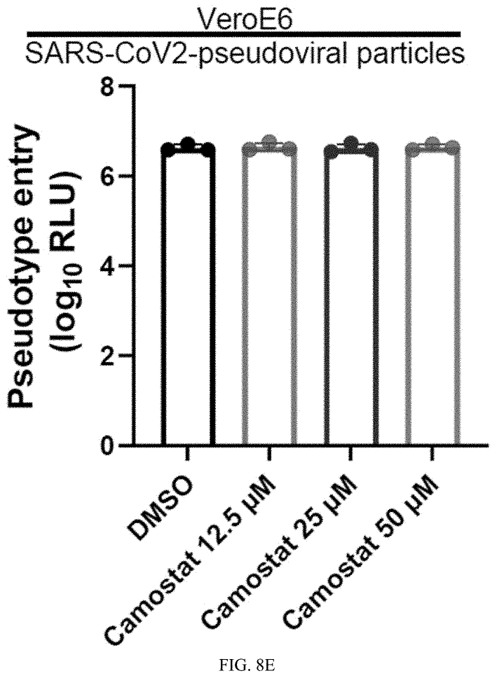

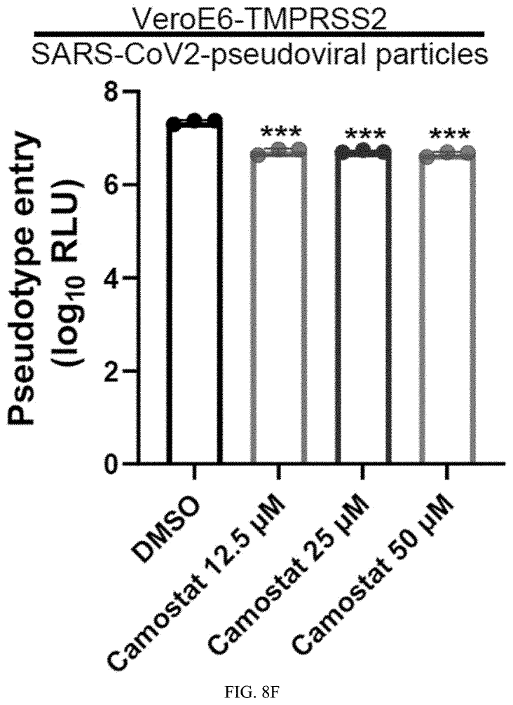

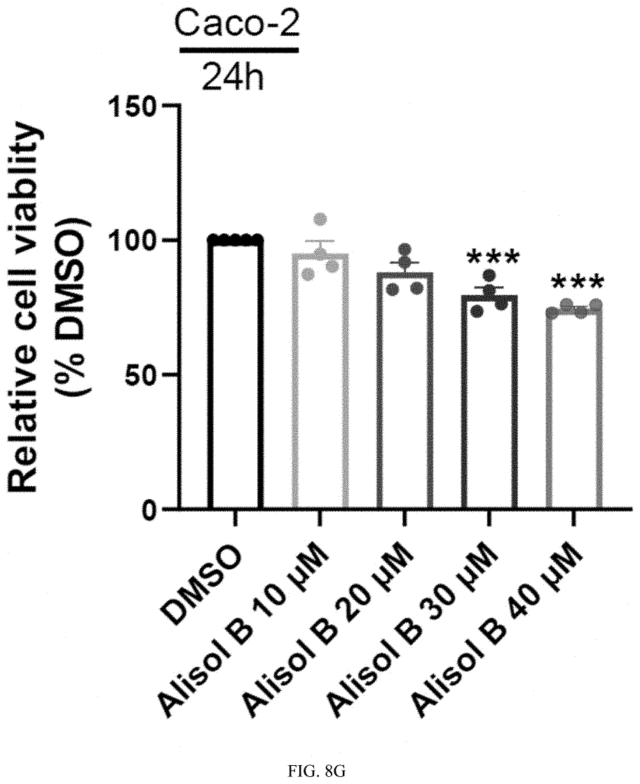

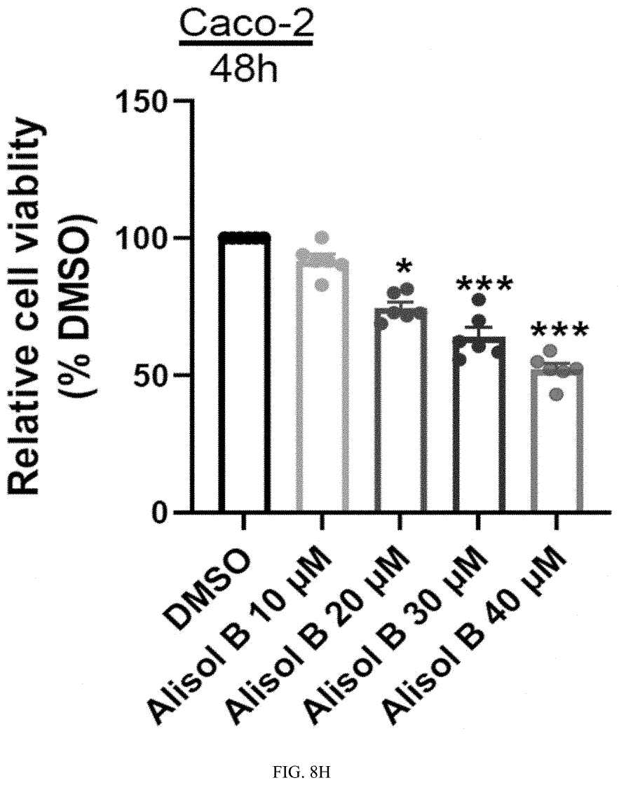

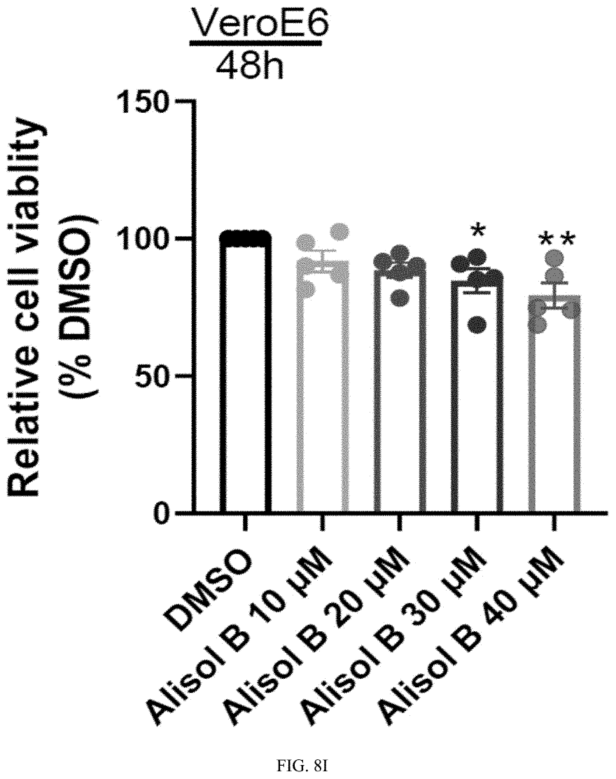

Previous studies indicate that the infiltrations of macrophages and T lymphocytes drive persistent alveolar inflammation in severe COVID-19 patients [16, 17]. Intriguingly, we found that Alisol B administration remarkably reduced the number of CD11b⁺ cells and CD3⁺ cells in the hamster lungs, indicating the inhibitions of the infiltration of macrophages and T cells respectively. Importantly, Alisol B treatment significantly increased the number of IgM⁺ B cells in the hamster lung tissues with the infection of SARS-CoV-2 and SARS-CoV-2 delta variant. Those results suggest that Alisol B could enhance the humoral immunity against CoVs infection (FIGS. 5A-5D, 5E-5H). We further verified the immunomodulation activity of Alisol B by adopting human lymphocytes in vitro. Alisol B treatment dose-dependently inhibited T cell proliferation and IL-17 secretion (FIGS. 6A-6C). Alisol B also reduced the number of CD11b⁺ macrophages in a dose-dependent manner (FIGS. 6I-6M, 9E-9N). Moreover, Alisol B administration dramatically boosted the B220⁺ B cells (FIGS. 6G and 6N). These results suggest that Alisol B confers humoral immunity against SARS-CoV-2 and SARS-CoV-2 delta variant challenge, while reduces T cells and macrophages associated inflammatory dysregulations.

It should be understood that the examples and embodiments described herein are for illustrative purposes only and that various modifications or changes in light thereof will be suggested to persons skilled in the art and are to be included within the spirit and purview of this application. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

1 Organization, W. H., Coronavirus Disease (COVID-19) Situation Reports, <see Worldwide website: who.int/emergencies/diseases/novel-coronavirus-2019/situation-reports>2020.
2. Russell, C. D., J. E. Millar, and J. K. Baillie, Clinical evidence does not support corticosteroid treatment for 2019-nCoV lung injury. The Lancet, 2020. 395(10223): p. 473-475.
3. Hung, I. F.-N., et al., SARS-CoV-2 shedding and seroconversion among passengers quarantined after disembarking a cruise ship: a case series. The Lancet. Infectious diseases, 2020. 20(9): p. 1051-1060.
4. Harvey, W. T., et al., SARS-CoV-2 variants, spike mutations and immune escape. Nature Reviews Microbiology, 2021. 19(7): p. 409-424.
5. Twohig, K. A., et al., Hospital admission and emergency care attendance risk for SARS- CoV-2 delta (B.1.617.2) compared with alpha (B.1.1.7) variants of concern: a cohort study. The Lancet Infectious Diseases.
6. Challen, R., et al., Risk of mortality in patients infected with SARS-CoV-2 variant of concern 202012/1: matched cohort study. BMJ, 2021. 372: p. n579.
7. Matsuda, H., et al., Effects of sesquiterpenes and triterpenes from the rhizome of Alisma orientale on nitric oxide production in lipopolysaccharide-activated macrophages: absolute stereostructures of alismaketones-B 23-acetate and-C 23-acetate. Bioorganic & medicinal chemistry letters, 1999. 9(21): p. 3081-3086.
8. Meng, Q., et al., Protective effects of alisol B 23-acetate from edible botanical Rhizoma alismatis against carbon tetrachloride-induced hepatotoxicity in mice. 2015. 6(4): p. 1241-1250.
9. Law, B. Y., et al., Alisol B, a novel inhibitor of the sarcoplasmic/endoplasmic reticulum Ca2+ ATPase pump, induces autophagy, endoplasmic reticulum stress, and apoptosis. 2010. 9(3): p. 718-730.
10. Jiang, Z.-Y., et al., A new triterpene and anti-hepatitis B virus active compounds from Alisma orientalis. Planta medica, 2006. 72(10): p. 951-954.
11. Chan, J. F.-W., et al., Simulation of the Clinical and Pathological Manifestations of Coronavirus Disease 2019 (COVID-19) in a Golden Syrian Hamster Model: Implications for Disease Pathogenesis and Transmissibility. Clinical infectious diseases: an official publication of the Infectious Diseases Society of America, 2020. 71(9): p. 2428-2446.
12. Riva, L., et al., Discovery of SARS-CoV-2 antiviral drugs through large-scale compound repurposing. Nature, 2020. 586(7827): p. 113-119.
13. Goncalves, D., et al., Antibodies against type I interferon: detection and association with severe clinical outcome in COVID-19 patients. Clinical & translational immunology, 2021. 10(8): p. e1327-e1327.
14. Grant, R. A., et al., Circuits between infected macrophages and T cells in SARS-CoV-2 pneumonia. Nature, 2021. 590(7847): p. 635-641.
15. Delorey, T. M., et al., COVID-19 tissue atlases reveal SARS-CoV-2 pathology and cellular targets. Nature, 2021. 595(7865): p. 107-113.
16. Peng, T., et al., A rationally designed rhodamine-based fluorescent probe for molecular imaging of peroxynitrite in live cells and tissues. Chemical science, 2016. 7(8): p. 5407-5413.
17. Yuan, S., et al., SREBP-dependent lipidomic reprogramming as a broad-spectrum antiviral target. Nature Communications, 2019. 10(1): p. 120.
18. Fan, M. Y., Liu, J., & Sun, R. (2020). Analysis on material basis and efficacy network of Huopo Xialing Decoction in treating damp pathogen stagnation of lung syndrome of early COVID-19. Chinese Traditional and Herbal Drugs, 51(9).
19. Din, M., Ali, F., Waris, A., Zia, F., & Ali, M. (2020). Phytotherapeutic options for the treatment of COVID-19: A concise viewpoint. Phytotherapy research: PTR, 34(10), 2431-2437. 20. The REMAP-CAP Investigators, Interleukin-6 Receptor Antagonists in Critically Ill Patients with Covid-19. New England Journal of Medicine 384, 1491-1502 (2021)).
21. Owen, D. R., C. M. N. Allerton, A. S. Anderson, L. Aschenbrenner, M. Avery, S. Berritt, B. Boras, R. D. Cardin, A. Carlo, K. J. Coffman, An oral SARS-CoV-2 Mpro inhibitor clinical candidate for the treatment of COVID-19. Science, eab14784 (2021).
22. A. Jayk Bernal, M. M. Gomes da Silva, D. B. Musungaie, E. Kovalchuk, A. Gonzalez, V. Delos Reyes, A. Martín-Quirós, Y. Caraco, A. Williams-Diaz, M. L. Brown, J. Du, A. Pedley, C. Assaid, J. Strizki, J. A. Grobler, H. H. Shamsuddin, R. Tipping, H. Wan, A. Paschke, J. R. Butterton, M. G. Johnson, C. De Anda, Molnupiravir for Oral Treatment of Covid-19 in Nonhospitalized Patients. New England Journal of Medicine 386, 509-520 (2021).
23. A. K. Singh, A. Singh, R. Singh, A. Misra, An updated practical guideline on use of molnupiravir and comparison with agents having emergency use authorization for treatment of COVID-19. Diabetes & Metabolic Syndrome: Clinical Research & Reviews 16, 102396 (2022).
24. E. Y. Dai, K. A. Lee, A. B. Nathanson, A. T. Leonelli, B. A. Petros, T. Brock-Fisher, S. T. Dobbins, B. L. Maclnnis, A. Capone, N. Littlehale, J. Boucau, C. Marino, A. K. Barczak, P. C. Sabeti, M. Springer, K. E. Stephenson, Viral Kinetics of Severe Acute Respiratory Syndrome Coronavirus 2 (SARS-CoV-2) Omicron Infection in mRNA-Vaccinated Individuals Treated and Not Treated with Nirmatrelvir-Ritonavir. medRxiv, 2022.2008.2004.22278378 (2022).
25. Q. Wang, Y. Guo, S. Iketani, M. S. Nair, Z. Li, H. Mohri, M. Wang, J. Yu, A. D. Bowen, J. Y. Chang, J. G. Shah, N. Nguyen, Z. Chen, K. Meyers, M. T. Yin, M. E. Sobieszczyk, Z. Sheng, Y. Huang, L. Liu, D. D. Ho, Antibody evasion by SARS-CoV-2 Omicron subvariants BA.2.12.1, BA.4 and BA.5. Nature, (2022).

We claim:

1. A method for prophylactic or responsive treatment of a human coronavirus infection or a symptom thereof in a subject, said method comprising administering an effective amount of 23-O-Acetylalisol B of Formula (I) to the subject, or a pharmaceutically acceptable salt, derivative, or prodrug thereof:

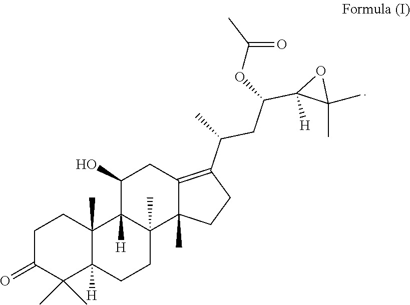

2. The method of claim 1, wherein the coronavirus is SARS-CoV-2.

3. The method of claim 2, wherein the SARS-CoV-2 is SARS-CoV-2 Alpha variant or SARS-CoV-2 Delta variant.

4. The method of claim 1, wherein the coronavirus is SARS-CoV.

5. The method of claim 1, wherein the coronavirus is MERS-CoV.

6. The method of claim 1, wherein the human coronavirus is a common human coronavirus selected from 229E, NL63, OC43, and HKU1.

7. The method of claim 1, wherein the subject is a human and has the coronavirus infection at the time of said administering.

8. The method of claim 1, wherein the subject is a human and has previously had the coronavirus infection at the time of said administering.

9. The method of claim 8, further comprising, prior to said administering, identifying the subject as having the coronavirus infection, wherein said identifying comprises assaying a biological sample obtained from the subject for the presence of coronavirus nucleic acid or coronavirus protein.

10. The method of claim 1, wherein the subject does not have the coronavirus infection at the time of said administering, and 23-O-Acetylalisol B is administered as prophylaxis.

11. The method of claim 1, wherein 23-O-Acetylalisol B is administered orally, intravascularly, nasally, rectally, parenterally, subcutaneously, or intramuscularly.

12. The method of claim 11, wherein 23-O-Acetylalisol B is administered orally.

13. The method of claim 1, wherein 23-O-Acetylalisol B reduces viral replication.

14. The method of claim 1, wherein 23-O-Acetylalisol B inhibits the infiltrations of CD11b-positive macrophages and CD3-positive T cells into the lung tissues.

15. The method of claim 1, wherein 23-O-Acetylalisol B decreases reactive oxygen species (ROS) and reactive nitrogen species (RNS) in lung tissues infected by SARS-CoV-2.

16. The method of claim 1, wherein 23-O-Acetylalisol B increases the proliferation and differentiation of human B cells.

17. The method of claim 16, wherein 23-O-Acetylalisol B increases IgM B cell populations in lung tissues.

18. The method of claim 1, wherein 23-O-Acetylalisol B inhibits the proliferation of human T lymphocytes and macrophages.

19. The method of claim 1, wherein 23-O-Acetylalisol B reduces the amount of IL-17, IFN-γ, IL6 and IP10 (CXCL10).

20. A method for treating an immune disorder in a subject, said method comprising administering an effective amount of 23-O-Acetylalisol B of Formula (I) to the subject, or a pharmaceutically acceptable salt, derivative, or prodrug thereof:

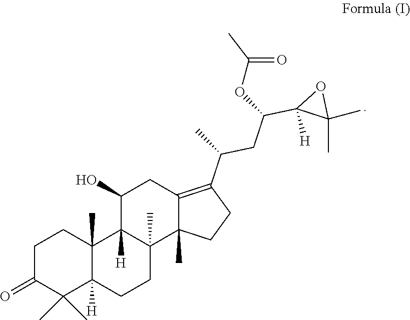

21. The method of claim 20, wherein the immune disorder is an autoimmune disorder selected from multiple sclerosis, systemic lupus erythematosus, or rheumatoid arthritis.

22. The method of claim 20, wherein 23-O-Acetylalisol B is administered orally, intravascularly, nasally, rectally, parenterally, subcutaneously, or intramuscularly.

23. The method of claim 22, wherein 23-O-Acetylalisol B is administered orally.

24. The method of claim 20, wherein 23-O-Acetylalisol B inhibits the infiltrations of CD3-positive T cells into the lung tissues.

25. The method of claim 20, wherein 23-O-Acetylalisol B inhibits the proliferation of human T lymphocytes.

26. The method of claim 20, wherein 23-O-Acetylalisol B reduces the amount of IL-17, IFN-γ, IL6 and IP10 (CXCL10).


SEQUENCE LISTING (5 sequences)

SEQ ID NO: 1 - DNA, 20 bp, Human coronavirus
  source 1..20: mol_type = genomic DNA
cgcatacagtcttrcaggct

SEQ ID NO: 2 - DNA, 24 bp, Human coronavirus
  source 1..24: mol_type = genomic DNA
gtgtgatgttgawatgacatggtc

SEQ ID NO: 3 - DNA, 28 bp, synthetic construct
  misc_feature 1..28: note = DNA probe for human coronavirus
  misc_feature 1: note = FAM tag
  misc_feature 28: note = Iowa Black FQ (IABkFQ) quencher
  source 1..28: mol_type = other DNA
ttaagatgtggtgcttgcatacgtagac

SEQ ID NO: 4 - DNA, 25 bp, Human coronavirus
  source 1..25: mol_type = genomic DNA
caaaaccttccctaagaaggaaaag

SEQ ID NO: 5 - DNA, 22 bp, Human coronavirus
  source 1..22: mol_type = genomic DNA
gctcctttggaggttcagacat